SEQUENCE LISTING (24 sequences)

SEQ ID NO: 1 - DNA, 20 bp, Homo sapiens
caacgtgttgcgattggtgt

SEQ ID NO: 2 - DNA, 20 bp, Homo sapiens
ccattctggcgtgtctccat

SEQ ID NO: 3 - DNA, 25 bp, Homo sapiens
cagaaaccataatctaccatggctg

SEQ ID NO: 4 - DNA, 20 bp, Homo sapiens
tgtgtttcttcggctgccca

SEQ ID NO: 5 - DNA, 29 bp, Homo sapiens
ccatatagactattggaaacacatgcgcc

SEQ ID NO: 6 - DNA, 30 bp, Homo sapiens
ctgtagttgcagttcaattgcttgtaatgc

SEQ ID NO: 7 - DNA, 20 bp, Homo sapiens
cagacgactatccagcgacc

SEQ ID NO: 8 - DNA, 20 bp, Homo sapiens
gcagtgaggattggagcact

SEQ ID NO: 9 - DNA, 20 bp, Homo sapiens
tacgtccgctgcttttgtct

SEQ ID NO: 10 - DNA, 20 bp, Homo sapiens
aacgcagaggctgctgttat

SEQ ID NO: 11 - DNA, 19 bp, Homo sapiens
ctgcaatgtttcaggaccc

SEQ ID NO: 12 - DNA, 20 bp, Homo sapiens
tcacgtcgcagtaactgttg

SEQ ID NO: 13 - DNA, 26 bp, Homo sapiens
atgagcaattaaatgacagctcagag

SEQ ID NO: 14 - DNA, 25 bp, Homo sapiens
cacacttgcaacaaaaggttacaat

SEQ ID NO: 15 - DNA, 20 bp, Homo sapiens
ggtgttgaggtaggtcgtcc

SEQ ID NO: 16 - DNA, 20 bp, Homo sapiens
cacacctgcatttgctgcat

SEQ ID NO: 17 - DNA, 20 bp, Homo sapiens
tgcatcggctacccaacttt

SEQ ID NO: 18 - DNA, 20 bp, Homo sapiens
acccgaccctgttccaattc

SEQ ID NO: 19 - DNA, 20 bp, Homo sapiens
gaccactaccagcagaacac

SEQ ID NO: 20 - DNA, 19 bp, Homo sapiens
gaactccagcaggaccatg

SEQ ID NO: 21 - DNA, 16 bp, Homo sapiens
cttgggccgcgtctcc

SEQ ID NO: 22 - DNA, 24 bp, Homo sapiens
gcaggaacccttataaccaaatcc

SEQ ID NO: 23 - DNA, 22 bp, Homo sapiens
agaggtcttcacgcagtccact

SEQ ID NO: 24 - DNA, 22 bp, Homo sapiens
ctctcctcactcacccttcctg

(19) United States
(12) Patent Application Publication
(10) Pub. No.: US 2020/0362305 A1
(43) Pub. Date: Nov. 19, 2020

(54) EFFICIENT INFECTION OF PRIMARY KERATINOCYTES BY HPV16

(71) Applicant: Board of Supervisors of Louisiana State University and Agricultural and Mechanical College, Baton Rouge, LA (US)

(72) Inventors: Martin SAPP, Shreveport, LA (US); Malgorzata BIENKOWSKA-HABA, Shreveport, LA (US)

(21) Appl. No.: 16/966,588

(22) PCT Filed: Feb. 13, 2018
(86) PCT No.: PCT/US2018/017926

§ 371 Date: Jul. 31, 2020

Related U.S. Application Data
(60) Provisional application No. 62/625,336, filed on Feb. 1, 2018.

(51) Int. Cl. C12N 5/071; C12N 7/00

(52) U.S. Cl. CPC C12N 5/0629; C12N 2533/90; C12N 2710/20021; C12N 7/00

(57) ABSTRACT

The claimed invention includes method comprising exposing HPV virions to an extra cellular matrix deposition outside of a mammalian body for a first set amount of time. The claimed invention also includes a virion subjected to such method. The claimed invention further includes a mammalian cell infected by a virion subjected to such method.

### CROSS REFERENCE TO RELATED APPLICATIONS/PRIORITY

The present invention claims priority to United States Provisional Patent Application Ser. No. 62/625,336 filed Feb. 1, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant Nos. R01A1081809; R01A1118904; R01DE025565 and R01CA211576 awarded by the National Institutes of Health. The government has certain rights in the invention.

### BACKGROUND OF THE INVENTION

High-risk HPV types are the infectious agents most commonly associated with human cancers such as, but not restricted to, cervical and oropharyngeal squamous cell carcinoma. The life cycle of these viruses is strictly dependent on the terminal differentiation process of keratinocytes. It is well established that tumor initiation requires deregulation of viral oncogene expression in the basal cell layer of the stratified epithelia, and continuous high-level oncogene expression is essential for tumor progression. While there is an understanding of processes leading to tumor progression, essentially no information is available regarding the early events resulting in increased oncogene expression. This can be attributed to the fact that no cell culture model has previously been available to study the immediate early events of the HPV life cycle. This is true despite more than 20 years of effort by a significant number of researchers in the field. While significant recent advances have been made, researchers were still unable to efficiently infect primary keratinocytes for the study of the complete viral life cycle. Instead, HPV-harboring keratinocytes either derived from lesions or established after transfection of the viral genome and waiting for outgrowth of immortalized clones have been used for most studies. However, establishment of these cell lines requires immortalization and high-level expression of viral oncogenes and thus bypasses the immediate early events of the viral life cycle. Events leading to immortalization after a natural infection could thus not be investigated. Furthermore, the need for immortalization severely restricted the number of HPV types to be studied and the genetic analyses of viral factors. For the foregoing reasons, there existed a pressing, but seemingly irresolvable need for efficient in infection of primary keratinocytes by HPV.

### SUMMARY OF THE INVENTION

Wherefore, it is an object of the present invention to overcome the above mentioned shortcomings and drawbacks associated with the current technology.

The inventors have now succeeded in developing an infection model that mimics immediate early events of the viral life cycle, is amenable to extensive genetic screens, can be expanded to essentially all HPV types, and allows the completion of the viral life cycle. This represents a significant technological advance that will enable the HPV research community to fill in huge gaps in our understanding of the regulation of oncogene expression and its deregulation in the early stages of tumor development. The inventors' model will also be extremely helpful in gaining a better understanding of the HPV life cycle. It allows a direct comparison of high-risk and low-risk HPV types for the first time. Furthermore, the model will be an essential model for the emerging field of studying the synergy of different pathogens in the development of tumors such as oropharyngeal squamous cell carcinoma.

The disclosed model system will not only be essential for the study of the HPV life cycle, it should also provide a useful platform for the testing and development of antivirals. Even though prophylactic vaccines are available, existing disease cannot be treated. Due to the long latency, HPV-induced disease will be present in the human population for decades to come even if everybody were to be vaccinated. Some HPV associated diseases, such as recurrent respiratory papillomatosis caused by HPV11, have no treatment options other than debilitating surgical procedures. The inventors' model is believed to be the first and only cell culture model allowing drug development for HPV11-induced disease.

The invention includes forming drug screens for HPV antivirals, including low risk HPV types such as HPV11 that cause significant morbidity and mortality (recurrent respiratory papillomatosis is one example).

Herein, the inventors disclose a novel infection model that achieves highly efficient infection of primary keratinocytes with human papillomavirus type 16 (HPV16). This cell culture model does not depend on immortalization and is amenable to extensive genetic analyses. In monolayer cell culture, the early but not late promoter was active and yielded a spliced viral transcript pattern similar to HPV16-immortalized keratinocytes. However, relative levels of the E8\hat{ }E2 transcript increased over time post infection suggesting the expression of this viral repressor is regulated independently of other early proteins and that it may be important for the shift from the establishment to the maintenance phase of the viral life cycle. Early and late promoter were strongly activated when infected cells were subjected to differentiation by growth in methylcellulose. When grown as organotypic raft cultures, HPV16-infected cells supported the completion of the viral life cycle. Intriguingly, RNA sequencing revealed a surprisingly small number of host genes deregulated in HPV16-infected compared to -immortalized keratinocytes. pRb- and p53-regulated pathways were affected in both cases. However, other pathways deregulated in immortalized keratinocytes were not altered in infected cells. As a proof of principle that the infection system may be used for genetic dissection of viral factors, we analyzed E1, E6 and E7 translation termination linker mutant virus. E1, but not E6 and E7, is essential to establish infection. Furthermore, E6 but not E7 is required for episomal genome maintenance. Primary keratinocytes infected with wild type HPV16 immortalized whereas keratinocytes infected with E6 and E7 knockout virus began to senesce 25 to 35 days post infection. The novel infection model provides a powerful genetic tool to study the role of viral proteins throughout the viral life cycle, but especially for immediate early events and enables the comparison of low- and high-risk HPV types in the context of infection.

The present invention is directed to methods, apparatuses, kits, and organisms that satisfy the above shortcomings and drawbacks.

The present invention also relates to a method comprising exposing HPV virions to an extra cellular matrix (ECM) deposition outside of a mammalian body for a first set amount of time. An additional embodiment includes exposing a mammalian cell to the HPV virion for a second set amount of time. An additional embodiment includes wherein the mammalian cell is a human cell. An additional embodiment includes the mammalian cell is a keratinocyte. An additional embodiment includes wherein the keratinocyte is a primary keratinocyte. An additional embodiment includes wherein the HPV virion is one of low-risk HPV and a high-risk HPV. An additional embodiment includes wherein the HPV virion a low-risk HPV. An additional embodiment includes wherein the low-risk HPV is one of HPV-6, HPV-11, HPV-40, HPV-42, HPV-43, HPV-44, HPV-53, HPV-54, HPV-61, HPV-72, HPV-73, and HPV-81. An additional embodiment includes wherein the HPV virion is a high-risk HPV. An additional embodiment includes wherein the high-risk HPV is one of HPV-16, HPV-18, HPV-31, HPV-33, HPV-35, HPV-39, HPV-45, HPV-51, HPV-52, HPV-56, HPV-58, HPV-59, and HPV-68. An additional embodiment includes wherein the ECM deposition includes depositions secreted by keratinocytes. An additional embodiment includes wherein the first set amount of time is at least two hours. An additional embodiment includes wherein the second set amount of time is one of greater than 2 days, less than 30 days, and between 2 days and 30 days. An additional embodiment includes wherein the second set amount of time is one of greater than 5 days, less than 7 days, and between 5 days and 7 days. An additional embodiment includes wherein the second set amount of time is sufficient for the mammalian cells to reach confluency. An additional embodiment includes the step of inducing differentiation of mammalian cells.

The disclosed invention further relates to an HPV virion subjected to the method of exposing the HPV virion to an ECM deposition outside of a mammalian body for a first set amount of time.

The disclosed invention further relates to a mammalian cell subjected to the method of exposing HPV virions to an ECM deposition outside of a mammalian body for a first set amount of time exposing the mammalian cell to the HPV virions for a second set amount of time. An additional embodiment includes wherein the mammalian cell is a human cell. An additional embodiment includes wherein the mammalian cell is a human cell. An additional embodiment includes wherein the mammalian cell is a keratinocyte.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is two photomicrographs of EdU-labeled pseudogenome primary human foreskin keratinocytes (HFK) resulting from an embodiment of the disclosed invention;

FIG. 2 is a graph of percent infected cells shown in FIG. 1

FIG. 3 is a bar chart showing relative HPV16 E1\hat{ }E4 transcripts for direct binding of HPV16 virions to HFK (“HPV16”) and binding of HPV16 virions to HFK following the disclosed ECM-to-cell transfer (“HPV16+ECM”);

FIG. 4 is a bar chart showing the relative transcript levels of E7 and E1\hat{ }\hat{ }E4 respectively for HFK when left on the ECM depositions 2, 4, and 7 days;

FIG. 5 is a bar chart showing the relative transcript levels of E7 and E1\hat{ }\hat{ }E4 respectively for human tonsilar epithelial (HTE) when left on the ECM depositions 2, 4, and 7 days;

FIG. 6 is a bar chart showing relative transcript levels of wild type HPV16 and HPV16 virions harboring a translation termination linker (TTL) mutation in E1;

FIG. 7 is a bar chart showing relative transcript levels of various viral open reading frames (ORF) at two and seven days post infection;

FIG. 8 is a bar chart showing the ratio of viral transcript levels of HPV16-infected and -immortalized HFK for various ORFs;

FIGS. 9 and 10 are profiles of RNA isolated from HPV16-infected-HFK and immortalized HFK with relation to a mapping of various ORFs;

FIG. 11 is a graphical representation of splicing events;

FIG. 12 is a bar chart of relative expression of differentiation markers loricrin of monolayer and methylcellulose for control and HPV16-infected;

FIG. 13 is a Western blot of the results of FIG. 11;

FIG. 14 is a bar chart of relative transcript level of HPV16-infected monolayer and methylcellulose for E7 and E1 \hat{ }E4;

FIG. 15 is a bar chart of relative transcript level of HPV16-infected monolayer and methylcellulose for various ORFs;

FIG. 16 are RNA profiles and splicing events for HPV16-infected monolayer and methylcellulose;

FIG. 17 is a bar chart of relative transcript level of HPV16-immortalized monolayer and methylcellulose for E6/E7 and E1 \hat{ }E4;

FIG. 18 is a Southern blot analysis of viral genome;

FIG. 19 is a bar chart of relative transcript level of HPV16-immortalized and HPV16-infected for various ORFs;

FIG. 20 is a bar chart of HPV16/pEGFP for rafts 1, 2, and 3;

FIG. 21 is a HPV16-specific fluorescent in situ hybridization (FISH) in rafts derived from both HPV16-immortalized and -infected HFK;

FIG. 22 is an immunofluorescent staining for E1\hat{ }E4 protein in cells of the upper layers of raft tissues;

FIG. 23 is an immunofluorescent staining for L1 protein in cells of the upper layers of raft tissues;

FIG. 24 is an immunofluorescent staining for MCM7 in HPV16-infected and mock-infected cells;

FIG. 25 is an immunofluorescent staining for PCNA in HPV16-infected and mock-infected cells;

FIG. 26 is an immunofluorescent staining for p53 signal in HPV16-infected and mock-infected cells;

FIG. 27 is a bar chart of relative transcript level for E6 and E1\hat{ }E4 for infected HPV16 wild type and infected HPV16 mutant viruses harboring translation termination linkers in the E6 or E7 open reading frames.

FIG. 28 is a photograph of an OncoE6™ Cervical Test results of test for E6 protein presence in infected HPV16 wild type and infected HPV16 mutant viruses harboring translation termination linkers in the E6 or E7 open reading frames;

FIG. 29 is a Western blot of presence of E7 protein with immortalized HPV16, infected HPV16 wild type and infected HPV16 mutant viruses harboring translation termination linkers in the E7 open reading frame;

FIG. 30 is a bar chart of relative transcript level for E6 for infected HPV16 wild type and infected HPV16 mutant viruses harboring translation termination linkers in the E6 or E7 open reading frames at 27-33 dpi and p+1;

FIG. 31 is a bar chart of % β-actin for infected HPV16 wild type and infected HPV16 mutant viruses harboring translation termination linkers in the E6 or E7 open reading frames;

FIG. 32 is a bar chart of exonuclease 5 resistance measured for each of HPV16 DNA, 18S ribosomal DNA, and mitochondrial DNA for each of infected HPV16 wild type and infected HPV16 mutant viruses harboring translation termination linkers in the E6 or E7 open reading frames;

FIG. 33 is a bar chart showing relative HPV16 E1\hat{ }E4 transcripts for binding of HPV16 virions for to HFK for infected cells detached from virus-loaded ECM at day 2 and reseeded on ECM-coated dishes (“HFK reseeded”) and for infected cells maintained on virus-loaded ECM at day 2;

FIG. 34 is a bar chart showing the relative transcript levels of E7 and E1\hat{ }E4 respectively for HPV16 virion infected cells exposed to dishes coated with ECM depositions from spontaneously immortalized human keratinocytes (“HaCaT”), HeLa, and HFK cells respectively;

FIG. 35 is a bar chart showing relative transcript levels of THE cells for various viral open reading frames (ORF) at seven days post infection;

FIG. 36 is a table of splicing events; and

FIG. 37 is a table of a list of oligonucleotide sequences used in PCR reactions.

### DETAILED DESCRIPTION OF THE PREFERRED EMBODIMENTS

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

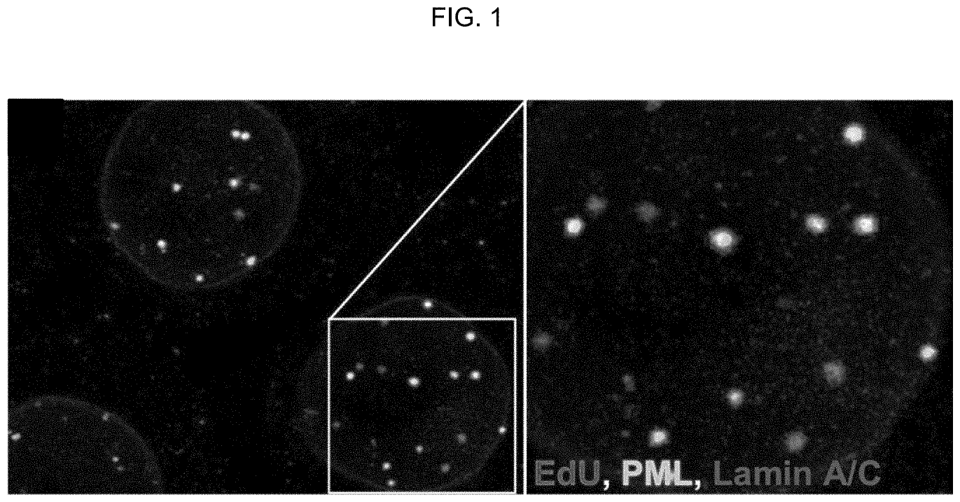

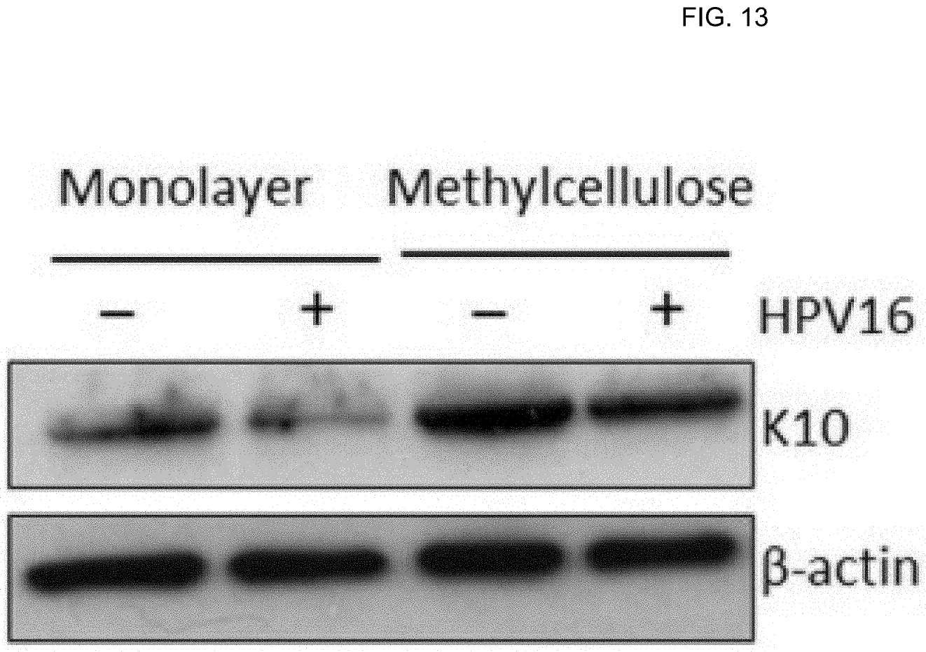

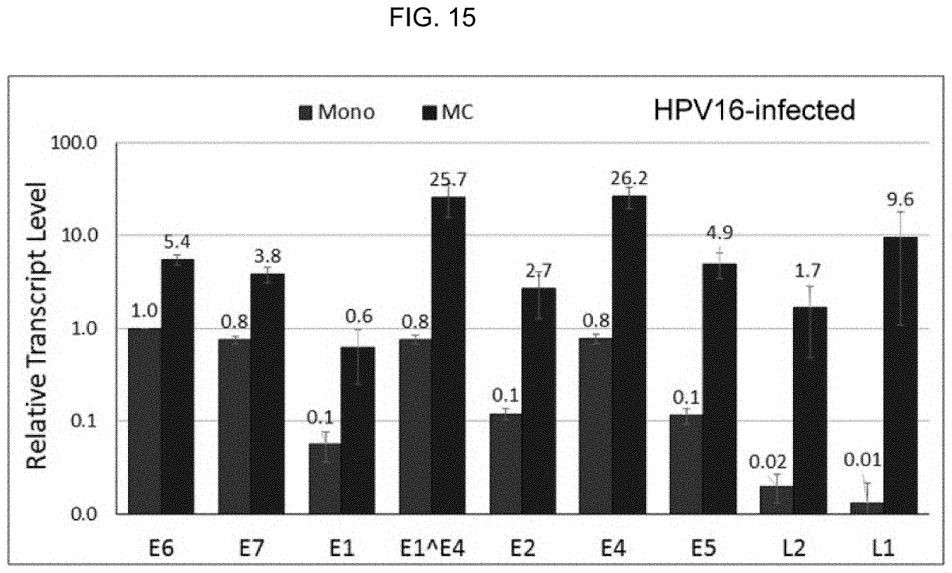

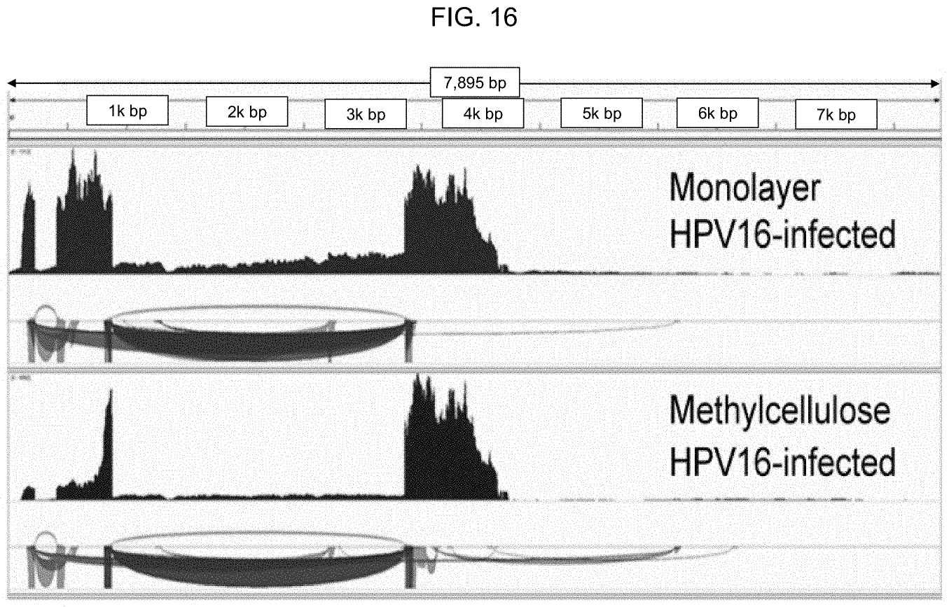

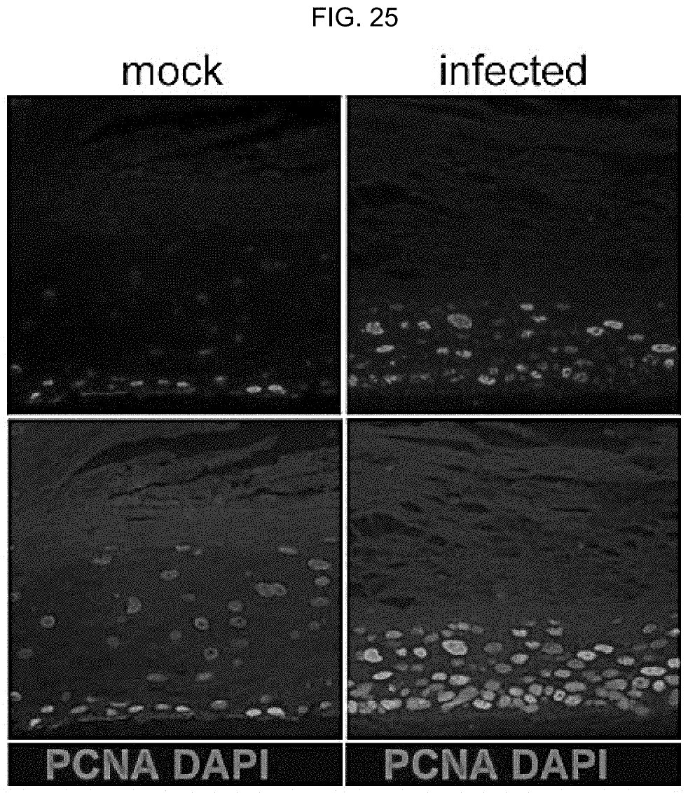

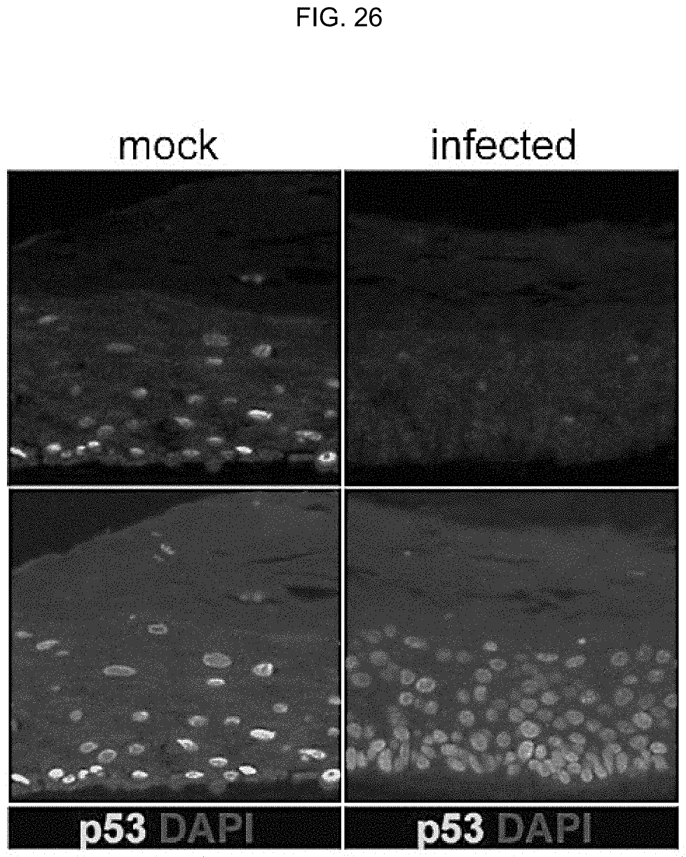

Turning now to FIGS. 1 through 37, a brief description concerning the various components of the present invention will now be briefly discussed.

High-risk HPV types such as HPV16 are the infectious agents most commonly associated with human cancers such as but not restricted to cervical and oropharyngeal squamous cell carcinoma. Approximately 5% of all human cancers can be linked to HPV infection, equating to approximately 700,000 new cancers a year. HPV encodes two major viral oncoproteins, E6 and E7, which drive immortalization and transformation of HPV infected cells. Their roles in cancer development can be mostly attributed to the inactivation of the p53 and pRb family of tumor suppressors, respectively. The viral oncogenes have been extensively studied over the past three decades, mainly using transfection models and recombinant retroviruses to express them in established and primary keratinocytes.

However, immortalization and transformation are not the default outcome of an HPV infection. Instead, oncogene expression is tightly regulated in a natural infection. The previous understanding of this regulation was very limited. The lack of knowledge was partly due to the fact that the HPV life cycle is strictly dependent on the terminal differentiation process of keratinocytes, making the studies technically difficult. A current view is that HPV gains access to stem and post stem cells of the basal layer through (micro)lesions by preferentially binding to the basement membrane (BM). After reaching the nucleus, it can be assumed that viral genome is initially amplified. This is based on the observation that up to several hundred copies of viral genome can be found in infected basal keratinocytes. After establishment of infection, the viral genome copy number is maintained in the basal compartment by maintenance replication. Viral transcription occurs at a low rate and it is assumed that the infection spreads by cell division. When HPV-harboring keratinocytes enter the terminal differentiation program, viral transcription is activated. Uninfected keratinocytes exit the cell cycle at this time and commit to terminal differentiation. However E7 protein, which negates the function of the pRb family members, allows HPV-harboring cells to maintain cell cycle competence. As a consequence, E1 and E2 protein in concert with the host cell replication machinery amplify the viral genome; a process that requires, through poorly understood mechanisms, activation of the DNA damage response and the function of the E4 and E5 viral proteins. Inactivation of p53 by E6 protein prevents cell cycle arrest due to unscheduled DNA replication. The viral life cycle is completed following structural (late)gene expression and assembly of progeny virions in highly differentiated cells of the uppermost layers of the stratified epithelium.

Most of the current knowledge is based on studying HPV-harboring keratinocytes either derived from lesions or established after transfection of the viral genome. However, establishment of these cell lines requires outgrowth of immortalized keratinocytes, which in turn depends on viral oncogene expression. According to current technology models, immortalization is associated with increased expression of E6 and E7. Therefore, HPV-harboring cells likely display deregulated viral oncogene expression and may not be suitable for the investigation of viral early promoter regulation after infectious entry. Thus, essentially no information is available regarding the early events that regulate viral oncogene expression in an HPV-infected basal cell; despite our detailed understanding of processes leading to tumor progression. Similarly, many assumptions about establishment of infection and shift to maintenance such as genome amplification during the establishment phase lack robust experimental support. This lack of knowledge can be attributed to the fact that no cell culture model has been available to study the immediate early events of the HPV life cycle, despite more than 20 years of effort by many researchers in the field and long felt need. While significant recent advances have allowed generation of virions using packaging cell lines or organotypic raft cultures, industry has been unable to infect primary keratinocytes efficiently for the study of the complete viral life cycle.

The inventors have now succeeded in developing an infection model that mimics immediate early events of the HPV life cycle. The infection model is amenable to extensive genetic screens, could be expanded to essentially all HPV types, and allows the completion of the viral life cycle. This represents a significant technological advance that will enable the HPV and cancer research community to fill in huge gaps in our understanding of the regulation of oncogene expression and its deregulation in the early stages of tumor development. The disclosed model will also be extremely helpful in gaining a better understanding of the HPV life cycle. It should allow a direct comparison of high and low-risk HPV types for the first time.

Results

Efficient HPV16 Infection of Primary Keratinocytes after ECM-to-Cell Transfer.

Direct binding of HPV16 to primary keratinocytes yields very inefficient infection rates for unknown reasons. However, the inventors were aware that HPV16 preferentially binds in vivo and in vitro to the basement membrane and the extracellular matrix (ECM) secreted by keratinocytes, respectively. The interactions with ECM-resident receptors such as LN332 and heparan sulfates were shown to be sufficient to induce conformational changes in viral capsid proteins that are important for infectious entry. Mutational analyses of receptor binding sites also suggested a unique contribution of LN332 to conformational shifts in capsid proteins.

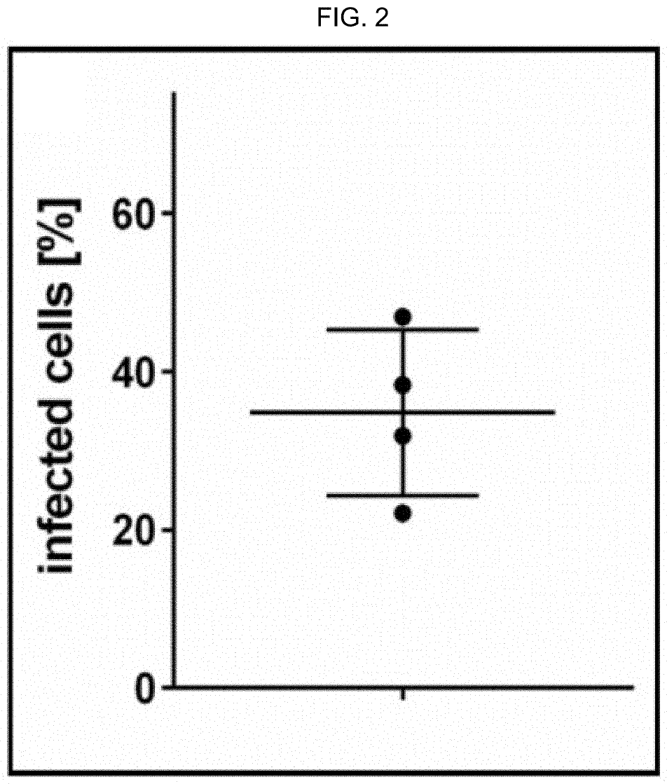

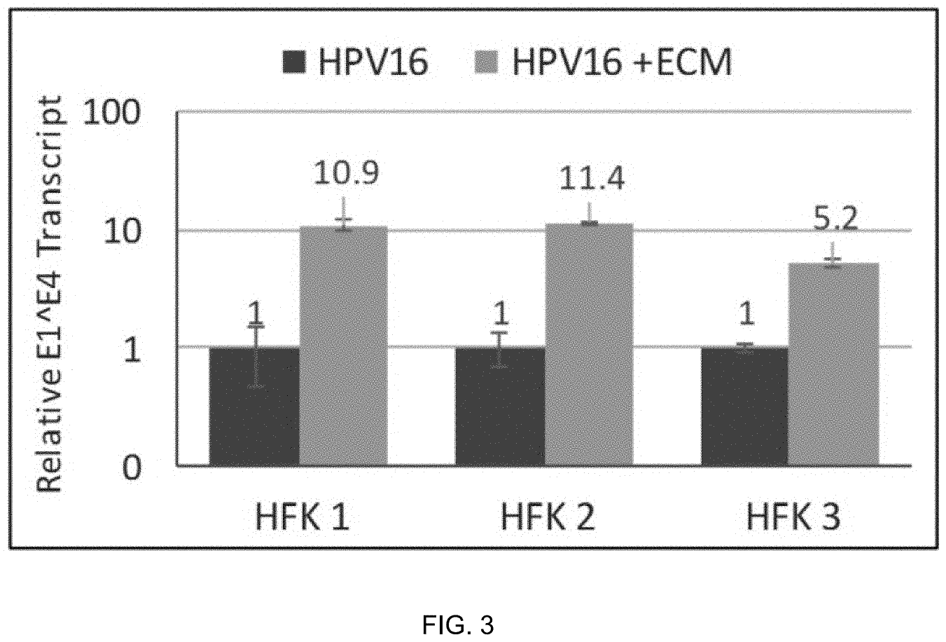

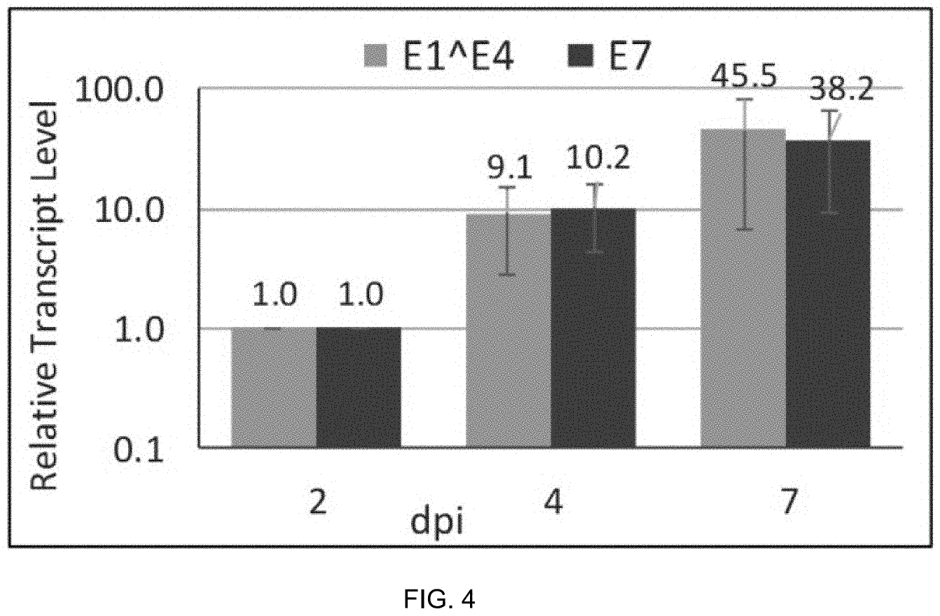

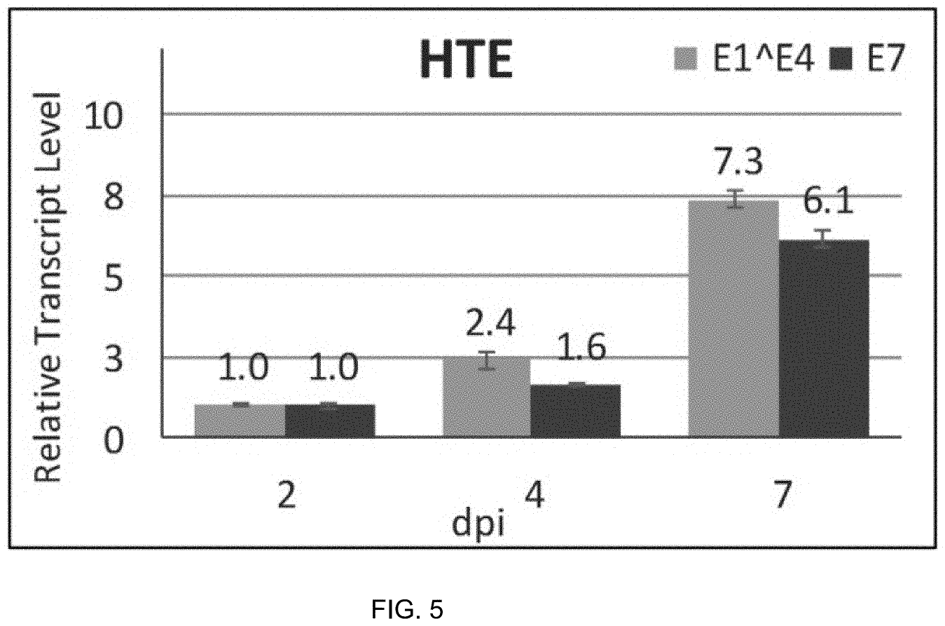

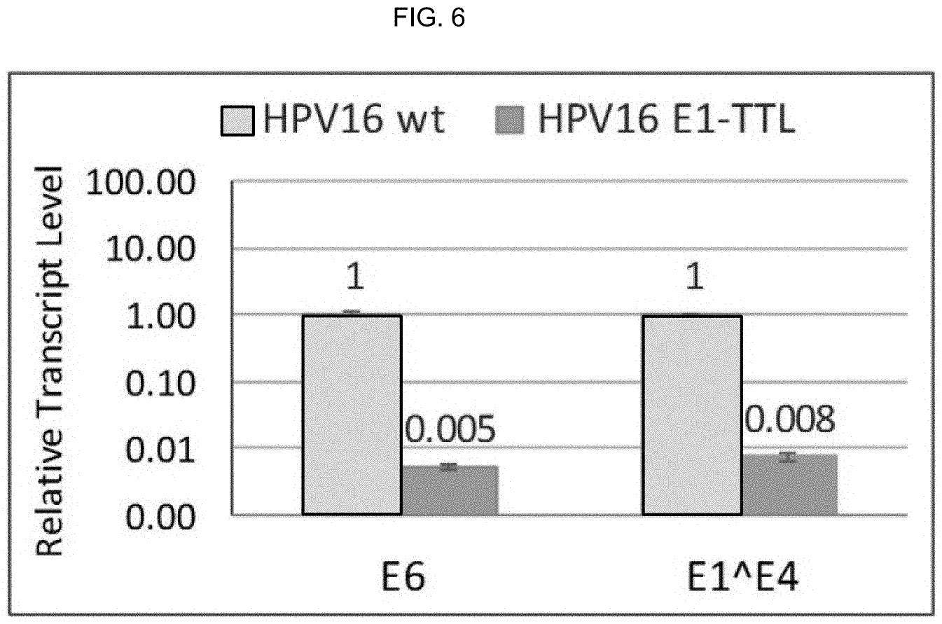

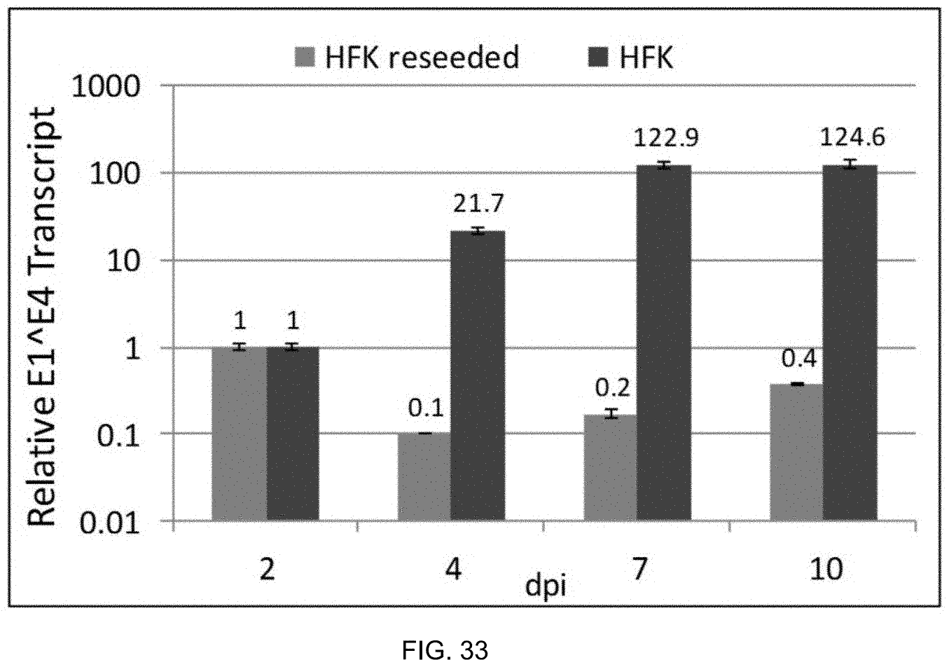

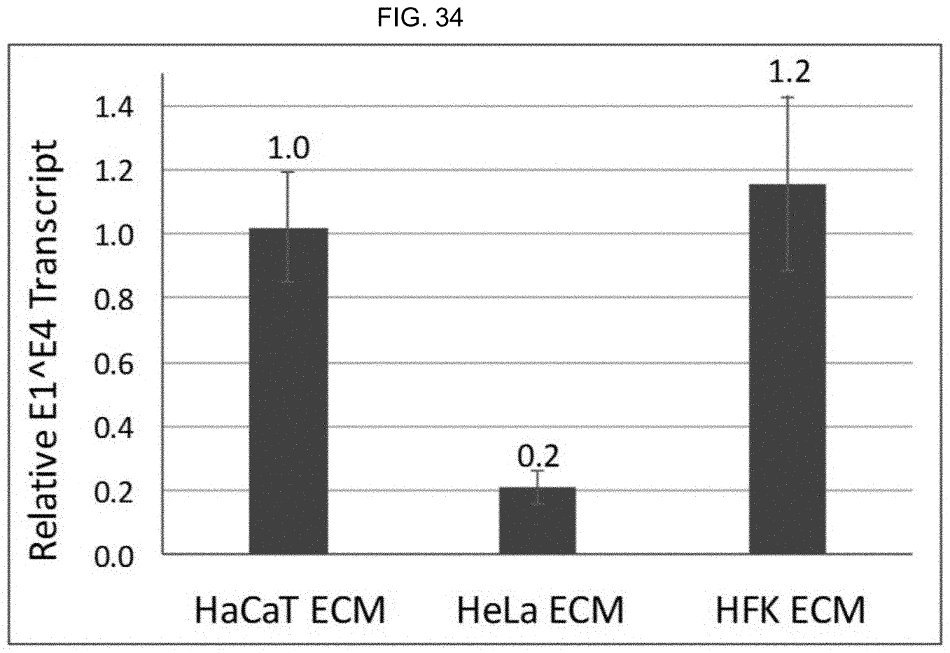

Based on these findings, the inventors hypothesized that pre-binding virions to ECM depositions would artificially mimic in vivo infection and improve infection of primary keratinocytes. To test this, HaCaT cells were grown in culture dishes for 48 h and subsequently removed by treatment with EDTA. Next, HPV16 viral particles generated using the 293TT packaging cell line were added to the ECM depositions left behind on the culture dish, incubated for 2 h and followed by seeding of primary keratinocytes. With this protocol, we were able to deliver EdU-labeled pseudogenome to the nuclei of close to 50% of primary human foreskin keratinocytes (HFK) at 40 hours post infection (hpi) using ECM-to-cell transfer (FIGS. 1 and 2). The inventors observed that HPV16 E1\hat{ }E4 transcripts were 10-fold higher following ECM-to-cell transfer of HPV16 virions as compared to direct binding to HFK at 72 hpi (FIG. 3). E7 and E1\hat{ }E4 transcript levels were further increased up to 50-fold when HFK were left on the ECM for 7 instead of 2 days with transcripts arising mostly from the early promoter (FIG. 4). Detaching infected cells from virus-loaded ECM at day 2 and reseeding on ECM-coated dishes did not yield higher transcript levels (FIG. 33). This finding suggests that increased transcript levels over time can be attributed to the continual delivery of viral genome rather than increased promoter activity. We were also able to efficiently infect primary human tonsilar epithelial (HTE) cells using this ECM-to-cell transfer to deliver viral genome (FIG. 5). HeLa cell secretions, which lack LN332, do not support efficient HPV16 infection (FIG. 34). This is in line with a suggestion that ECM-resident LN332 plays an important role in efficient ECM-to cell transfer. HPV16 virions harboring a translation termination linker (TTL) mutation in E1 failed to establish infection since viral transcripts were hardly detectable (FIG. 6) suggesting that E1 is essential for establishment of HPV16 infection and providing indirect support for the amplification of incoming viral genome.

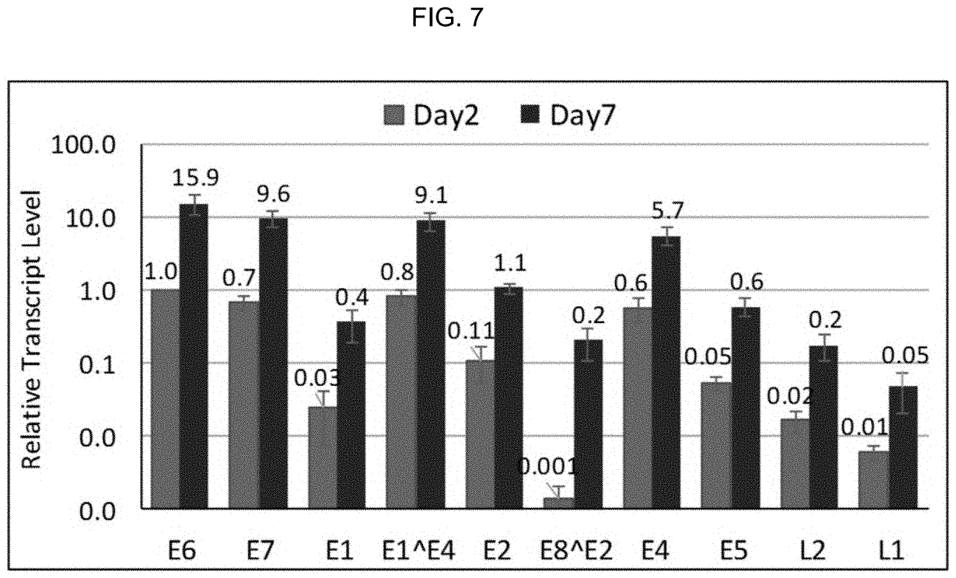

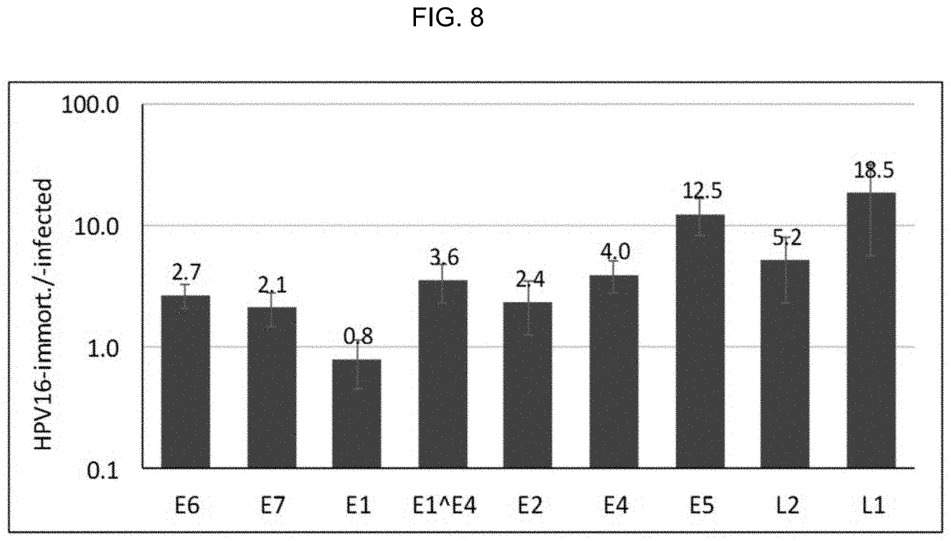

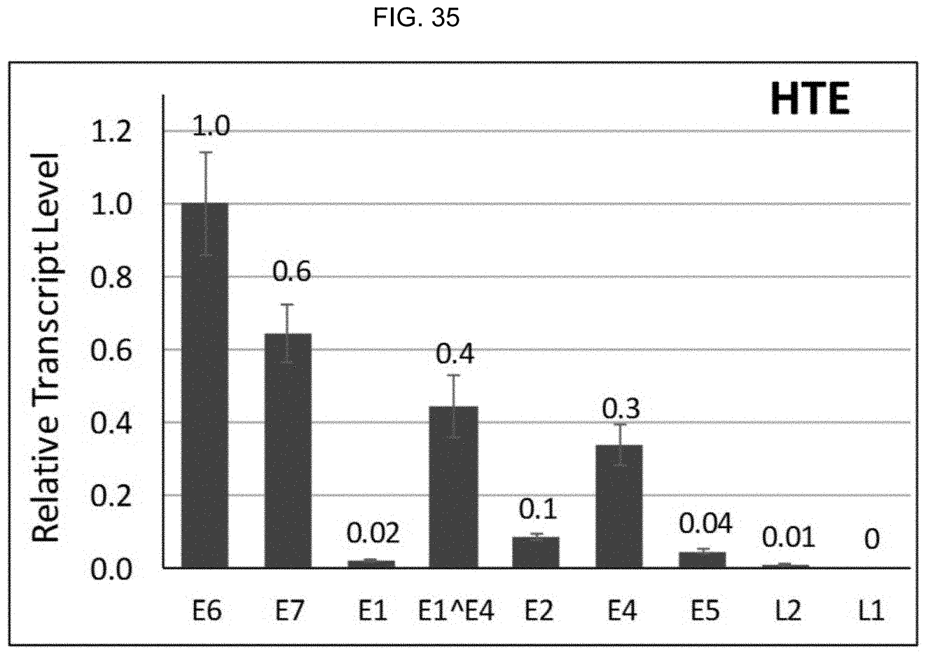

HPV16 early transcripts are transcribed from the early promoter p97 and are differentially spliced resulting in different quantities of viral open reading frames (ORF). The late promoter p670 is activated when infected keratinocytes enter the terminal differentiation program. As expected when primarily the p97 early promoter is active, the most abundant transcripts contained the E6, E7 and E4 ORFs, whereas the early E1, E5 and E2 transcripts were present at significantly lower levels (FIG. 7). The late L1 and L2 ORFs were essentially undetectable at two days post infection (dpi) of HFK and just barely reached the inventors' limit of detection at 7 dpi suggesting that the late promoter is under tight control in infected HFK. Similar results were obtained with HPV16-infected HTE (FIG. 35). When viral transcript levels between HPV16-infected and -immortalized HFK were compared, the inventors found that most early transcripts were present at 2- to 4-fold lower levels in HPV16-infected HFK (FIG. 8), with the exception of E1 encoding transcripts for which similar levels were found. The transcripts containing the late L1 and L2 ORFs as well as the E5 ORF were found at up to 20-fold lower levels in HPV16-infected compared to -immortalized HFK. The data imply a very tight control of the late promoter after HPV infection.

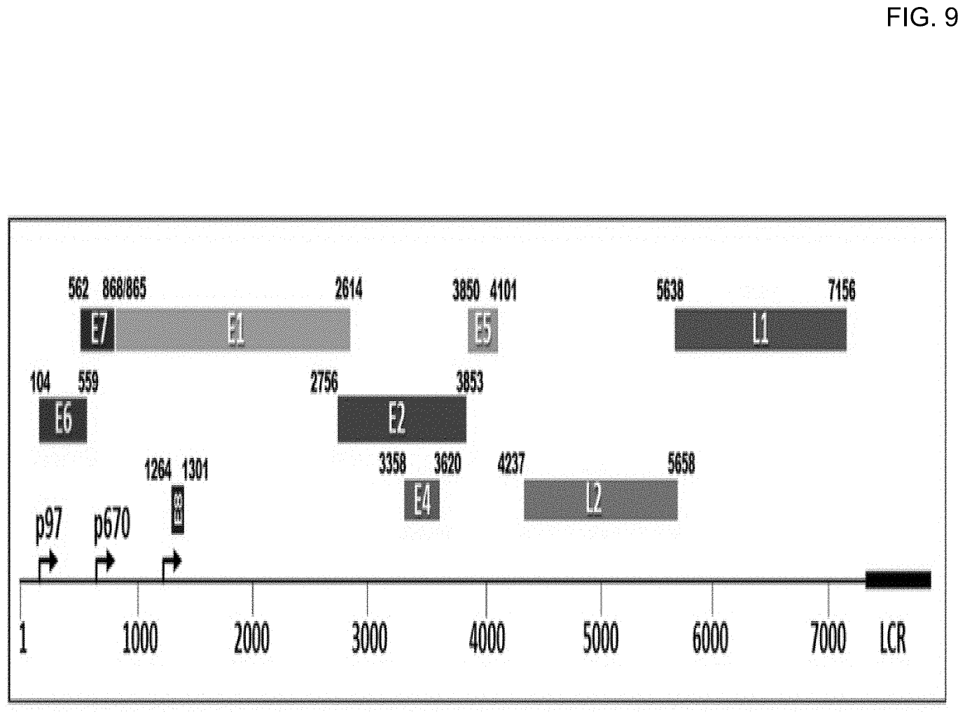

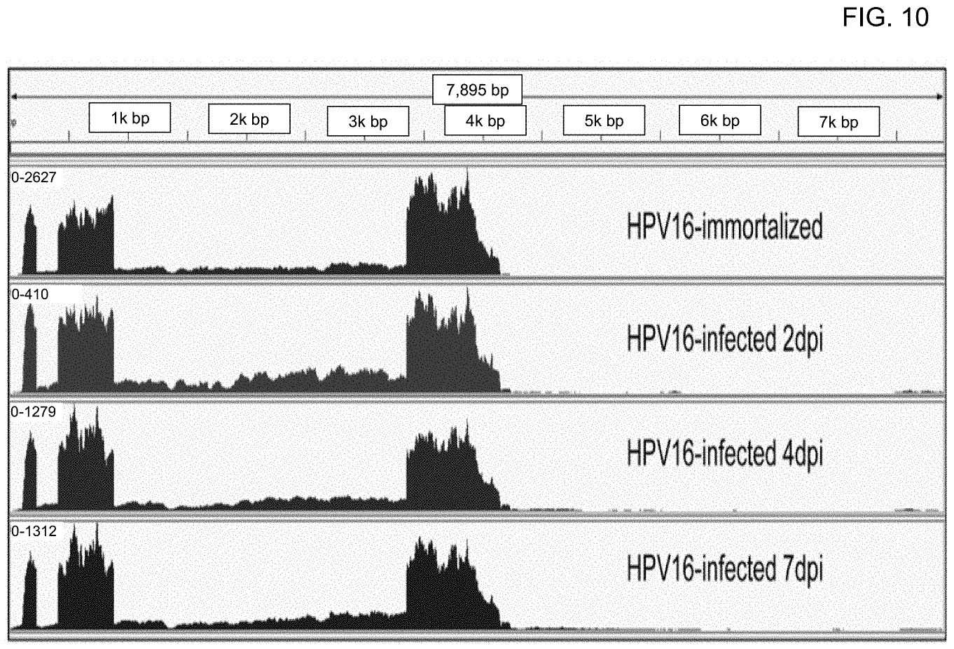

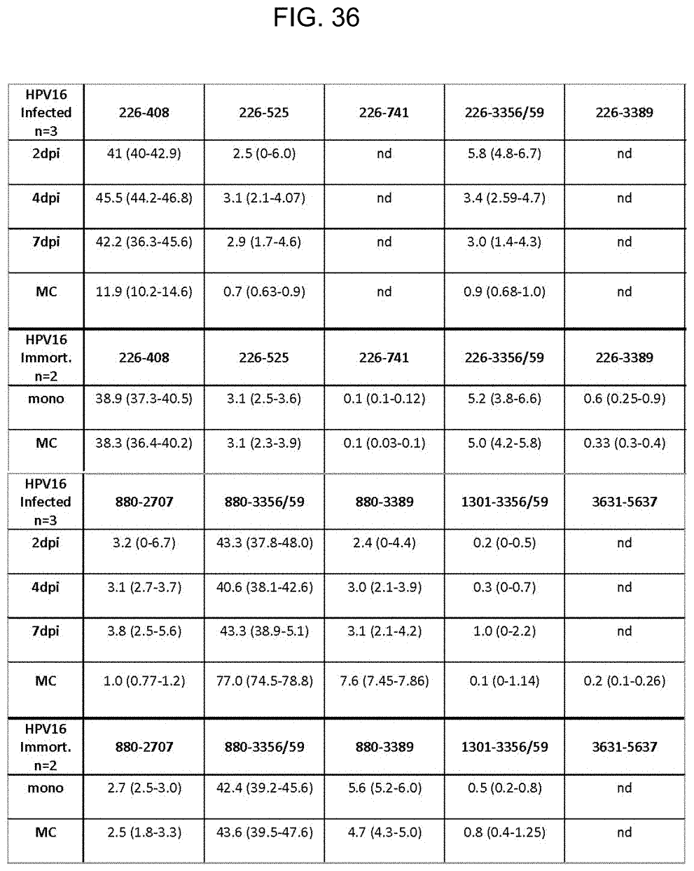

The inventors profiled RNA derived from HPV16-infected HFK at 2, 4 and 7 dpi using next generation sequencing (NGS) and compared the outcome to RNA isolated from HPV16-immortalized HFK. The overall profile of the viral transcripts isolated from HPV16-infected and -immortalized HFK is very similar despite differences in read depths, providing further support for the validity of the infection model (FIGS. 8 and 9). Two major splicing events use the 226 and the 409 (E6*I) and the 880 and 3358 (E1\hat{ }E4) splice acceptor and donor sites, respectively. Approximately 40 to 45% of all early transcripts are spliced at the 226/409, 40 to 43% use the 880/3358 splice donor and acceptor pair. Additional previously described junctions 226/526 (E611; 2.5-3.1%), 226/3358 (3-5.8%), 880/2709 (E2; 3.1-3.8%), and 880/3391 (2.4-3.1%), were also found at lower frequency (FIGS. 9 and 10; Table S1). In addition to the splice acceptor site at 3358, an alternative site at 3361 is being used at low frequency. The splice variant with E8\hat{ }E2 coding potential (1302/3358) is the only one, whose relative levels increase significantly over time post infection compared to other early transcripts (FIG. 36) suggesting that it may be important for a switch to maintenance replication and offering support for previous reports suggesting a repressive role for E8\hat{ }E2 (28-31). Less than 5% of the early transcripts have coding potential for full-length E6. The NGS results also confirm the low abundance of E1 and E2 encoding RNAs (FIG. 7). Some minor splice variants previously reported in HPV16-immortalized cells and confirmed by our analysis were not present in HPV16-infected cells (FIG. 36).

The Early and Late Promoters are Activated by Differentiation.

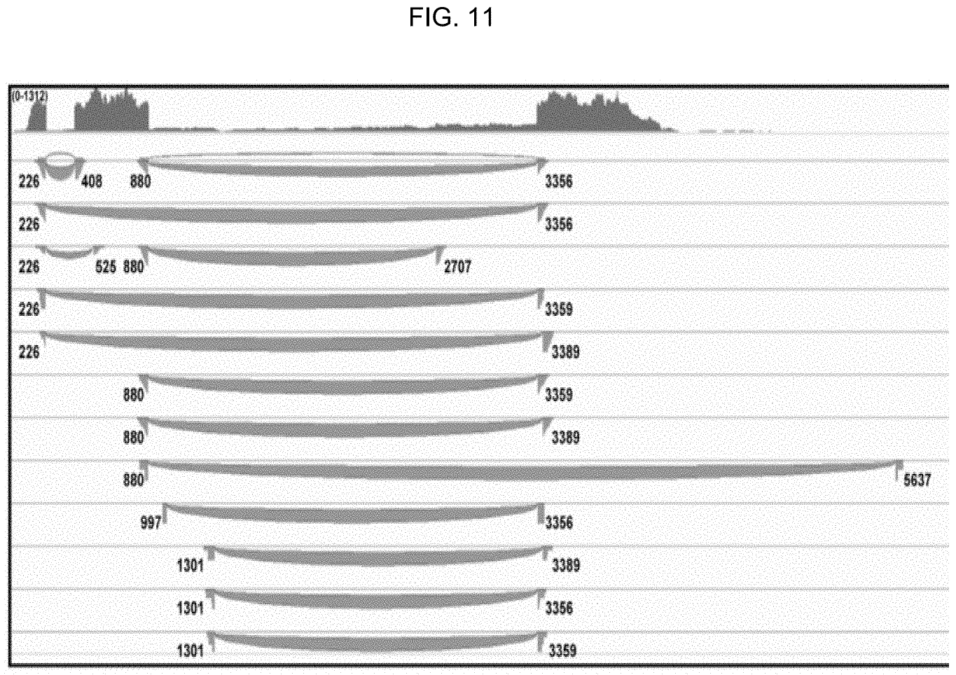

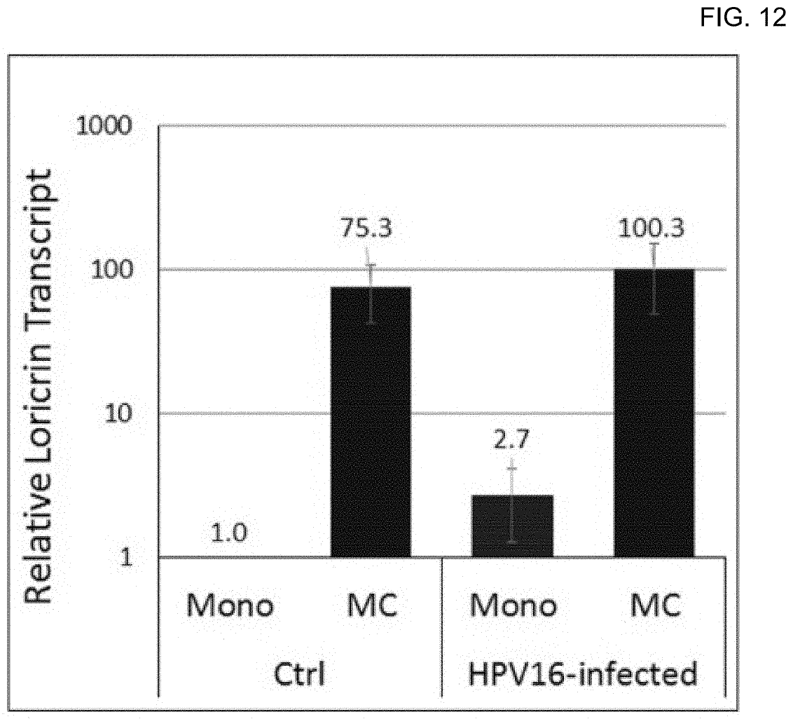

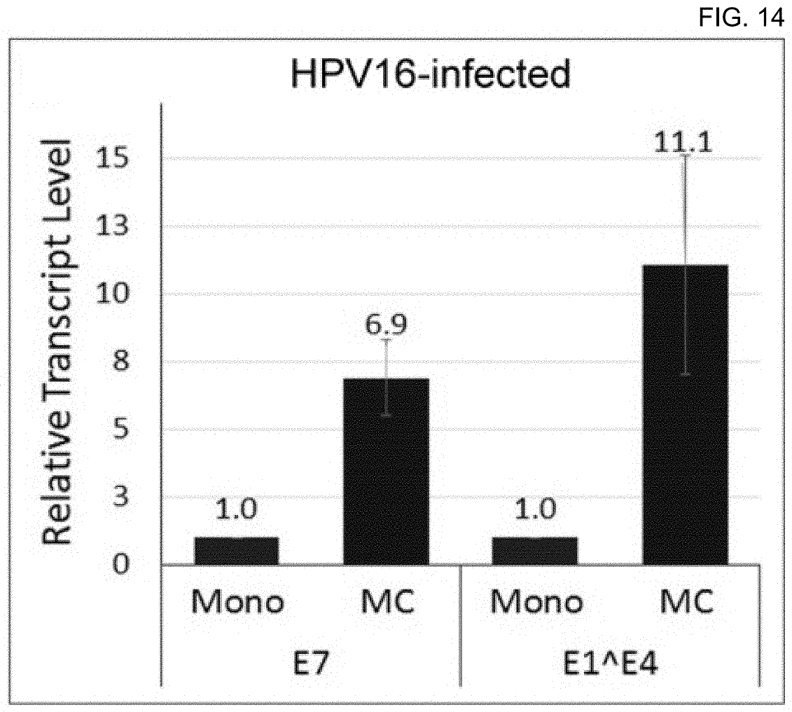

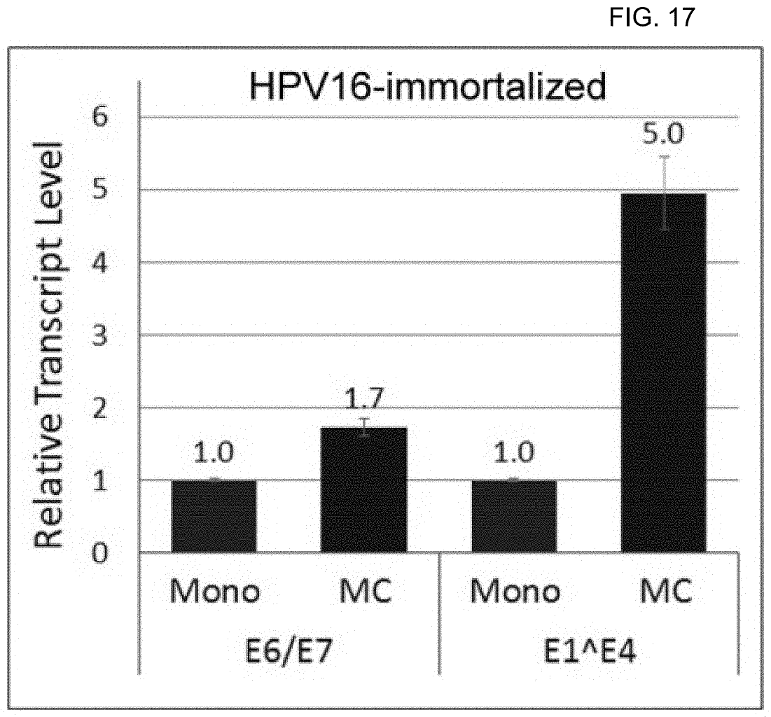

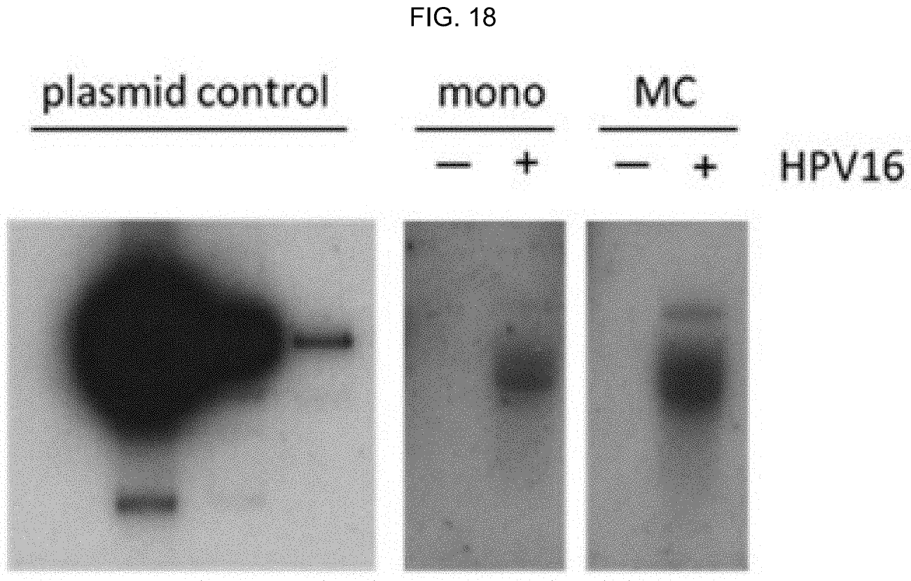

To test whether the incoming viral HPV16 genome is responsive to differentiation, the inventors subjected HFK infected for 5 days with HPV16 virions to growth in semi-solid methylcellulose (MC) media, which is established to induce differentiation of keratinocytes and to activate the viral late promoter. Differentiation was confirmed by increased expression of differentiation markers loricrin and keratin 10 by RT-qPCR (FIG. 11) and by Western blot (FIG. 12), respectively. Activation of the late promoter was observed by quantitative reverse transcription PCR (RT-qPCR) and confirmed by NGS giving rise to late L1- and L2-encoding transcripts (FIGS. 14-16). In addition, the early promoter was activated as evidenced by a 7-fold increase of early transcripts (FIG. 14). This was seen when HFK were grown in the presence and absence of the ROCK inhibitor. We would like to point out that the E1\hat{ }E4 transcript measured in FIG. 14 can arise from both the early and late promoter. In contrast, growth of HPV16-immortalized HFK in MC activated the late but only weakly the early promoter (FIG. 17). Southern blot analysis of viral genome also suggested increased viral genome levels after growth of HPV16-infected HFK in MC (FIG. 18). These data indicate that the viral genome delivered by HPV16 particles establishes infection and responds to differentiation. Furthermore, our data suggest that not only the late but also the early promoter responds to differentiation, thus providing the first experimental evidence of naturally infected lesions.

Organotypic Raft Culture of HPV16-Infected HFK.

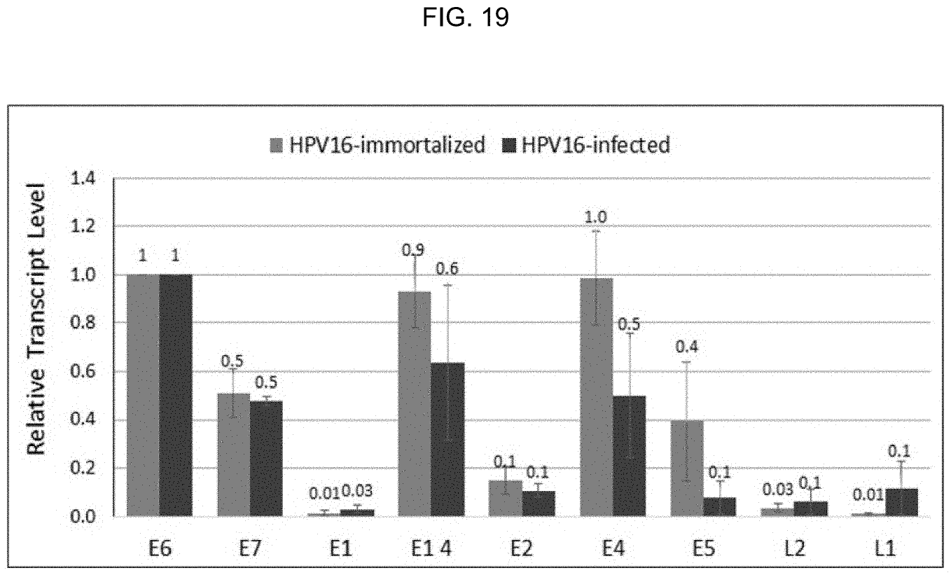

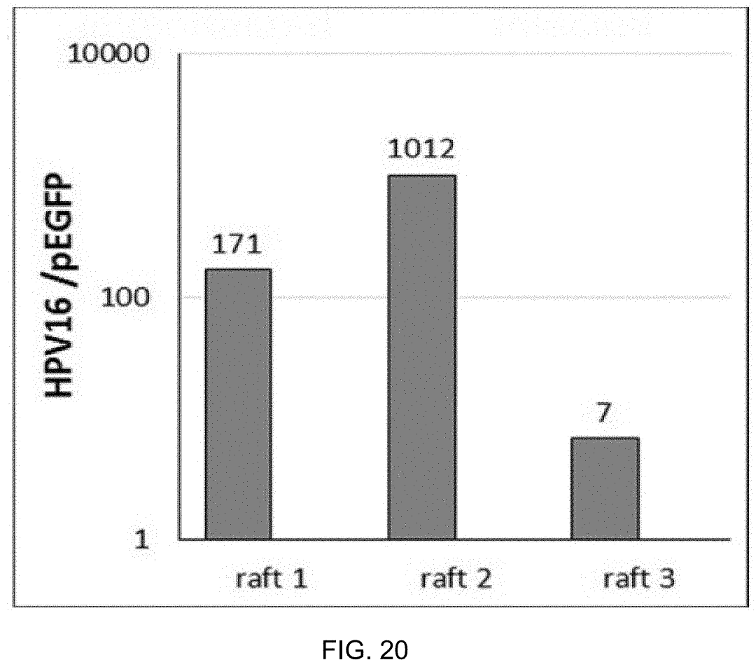

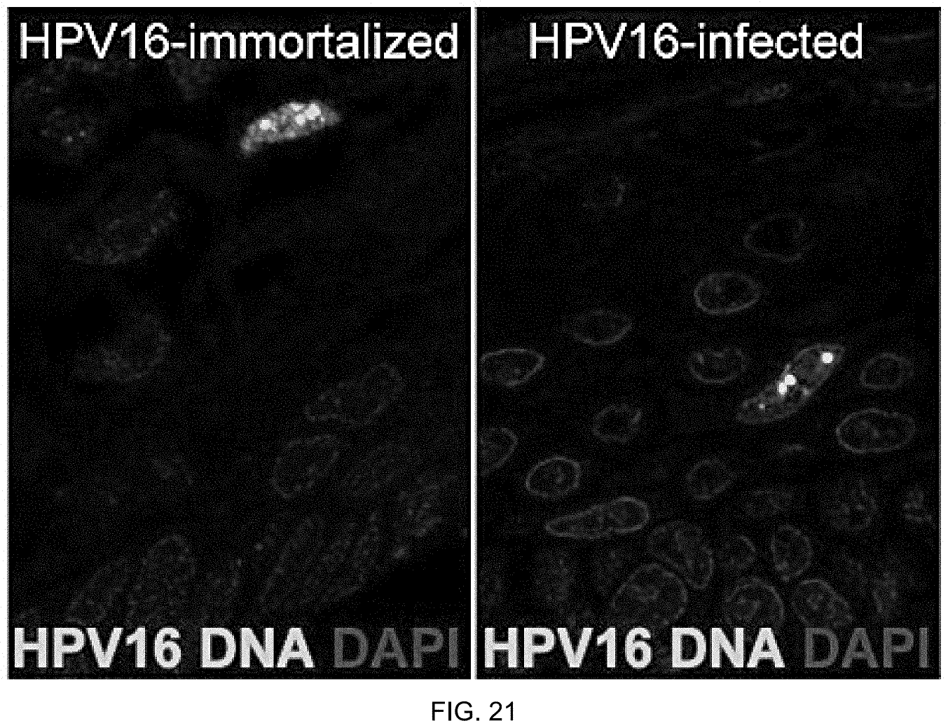

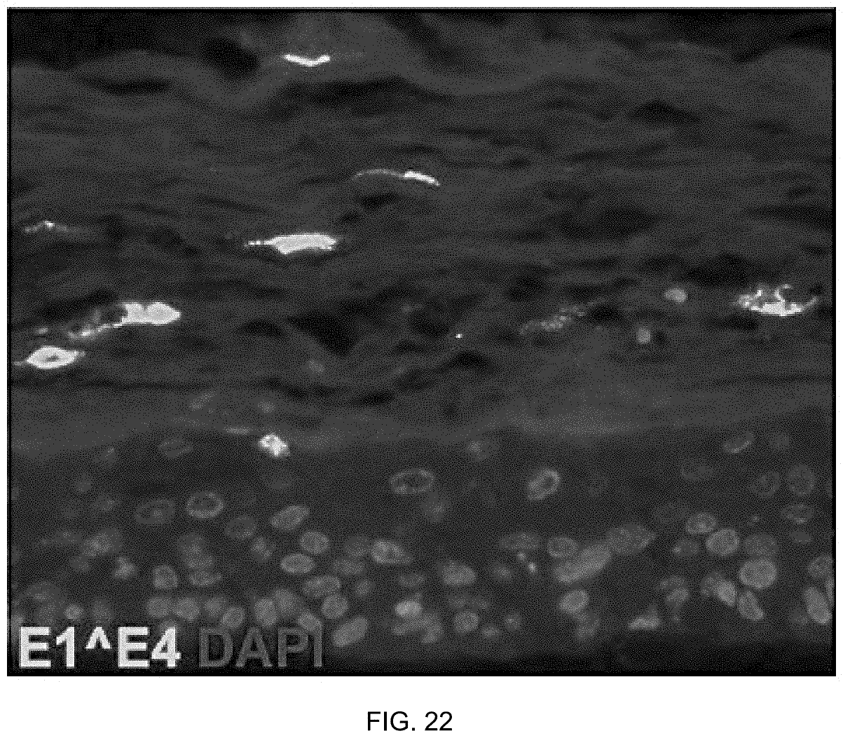

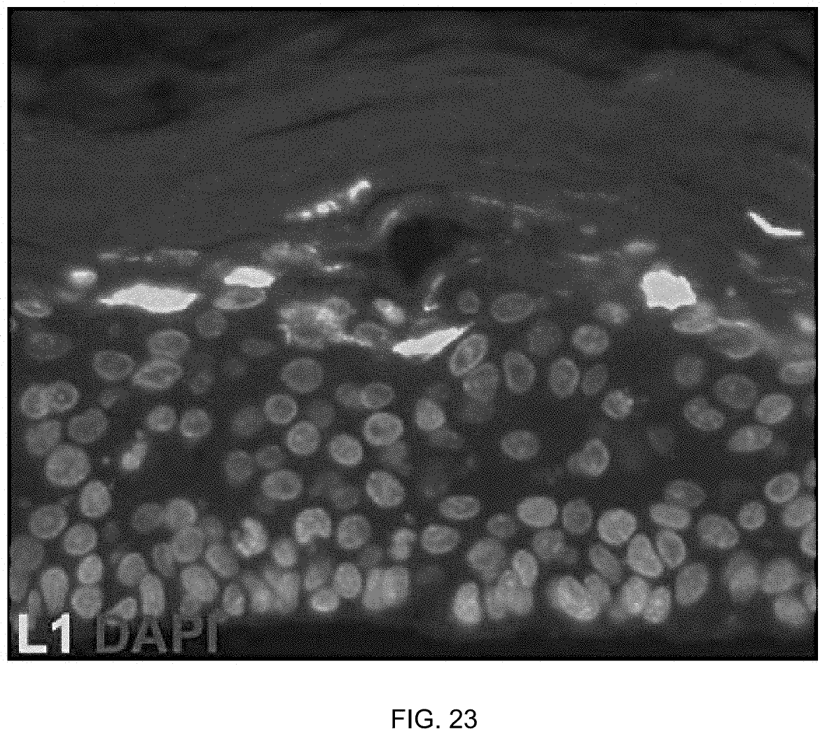

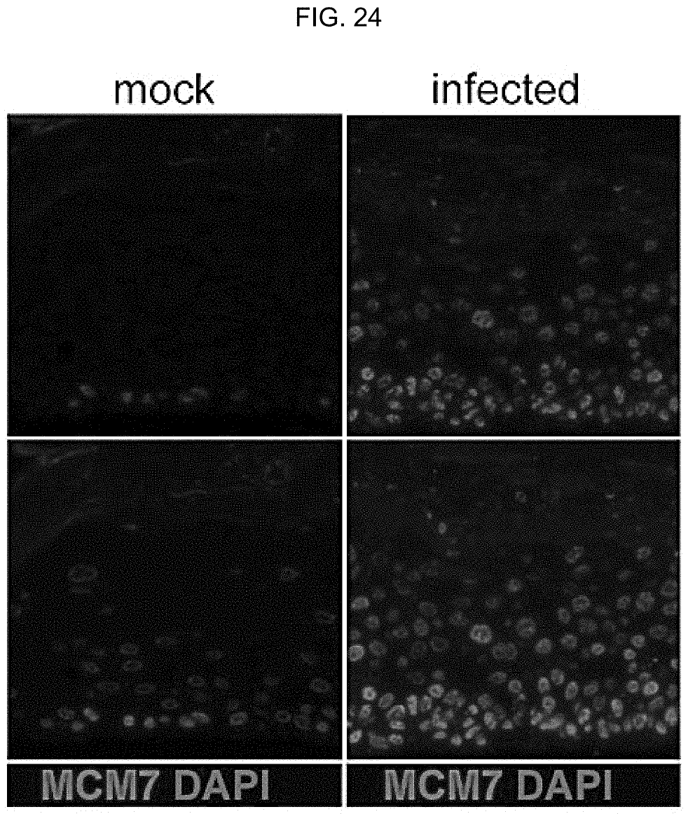

The inventors next subjected HFK infected for 5-7 days with HPV16 to organotypic raft cultures, which the inventors know to support completion of the viral life cycle. Uninfected and HPV16-immortalized HFK served as negative and positive controls, respectively. As shown in FIG. 19, both early and late transcripts were detectable in rafts and the expression profile of viral RNA isolated from rafts derived from infected and immortalized HFK were similar, albeit total viral RNA levels tended to be lower in rafts from HPV16-infected cells. We also observed that HPV16 genome was retained in the raft cultures, thereby suggesting replication of viral genome has occurred (FIG. 20). Indeed, HPV16-specific fluorescent in situ hybridization (FISH) identified cells with replication foci in rafts derived from both HPV16-immortalized and -infected HFK (FIG. 21). Immunofluorescent staining for E1\hat{ }E4 and L1 protein were positive in many cells of the upper layers of the raft tissues (FIGS. 22 and 23). Furthermore, markers of cell proliferation such as MCM7 and PCNA were present throughout the parabasal and spinous layers of the stratified epithelia and p53 signal was greatly diminished in HPV16-infected but not mock-infected cells (FIGS. 24-26). These results confirm our previous observation that most cells had been infected. Taken together, amplification of the viral genome and the presence of L1 protein suggest that the ECM-to-cell transfer infection model allows recapitulation of the complete viral life cycle.

E6 but not E7 is Essential for Genome Maintenance in Monolayer Cell Cultures.

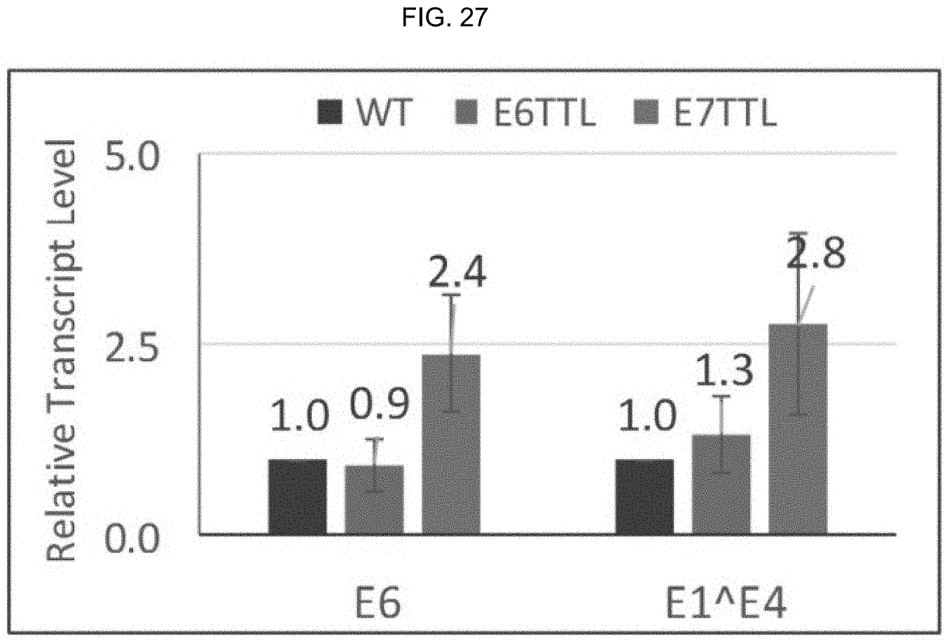

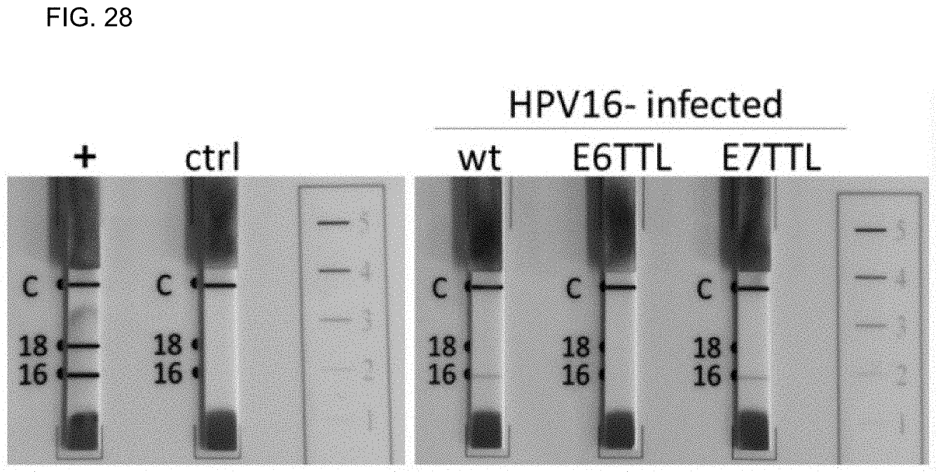

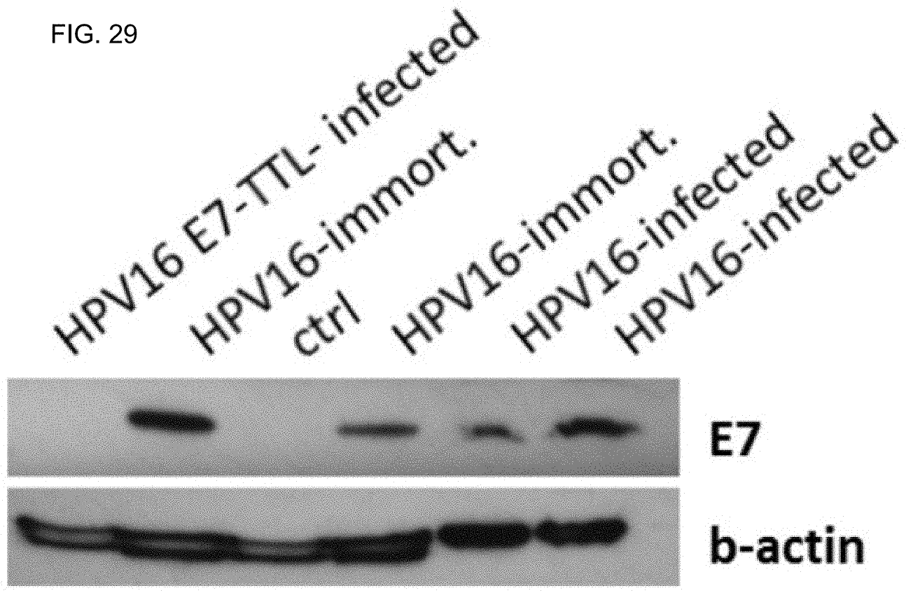

As proof of principle that the infection model is amenable to genetic analyses, the inventors generated HPV16 mutant viruses harboring translation termination linkers in the E6 and E7 open reading frames. Both mutant viruses established infection as evidenced by the presence of early transcripts (FIG. 27). We subjected extracts derived from HFK infected with respective wild type (wt) and mutant virus at 7 dpi to western blot analysis and a commercially available test for detection of E7 and E6 protein, respectively. E6 and E7 proteins were detected in HFK infected with wt HPV16 but were absent after infection with the respective mutant virus (FIGS. 28 and 29). As such, the inventors concluded that expression of E6 is not impaired by E7 knockout and vice versa.

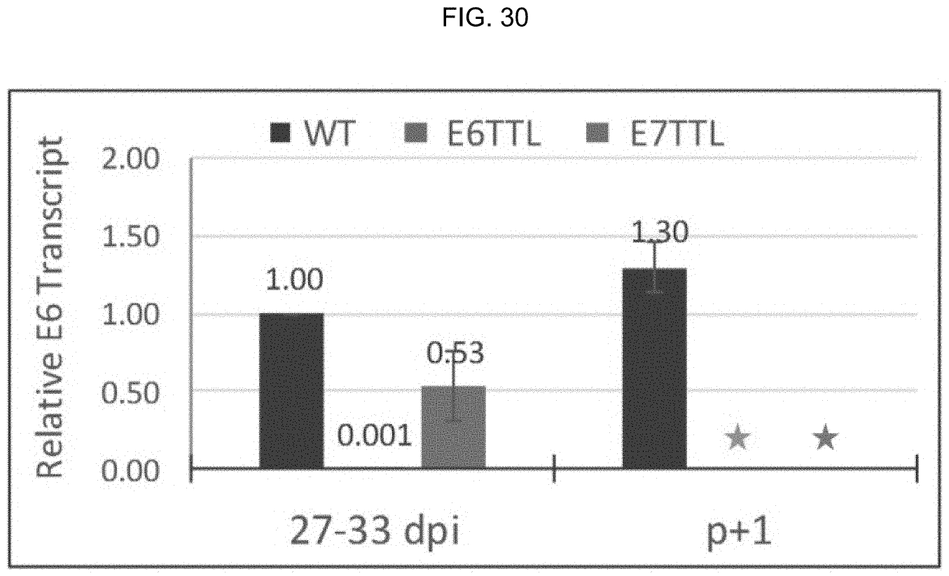

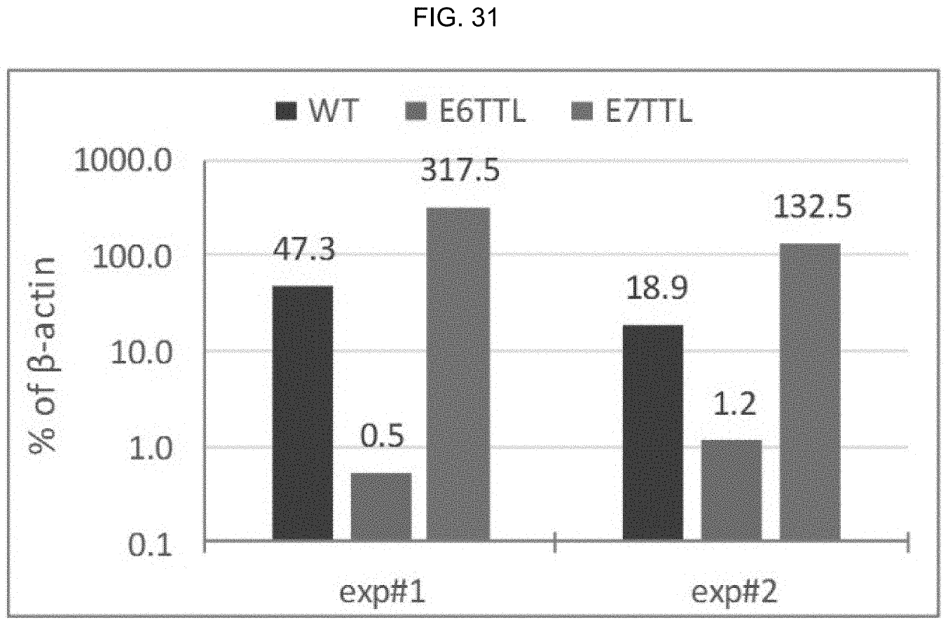

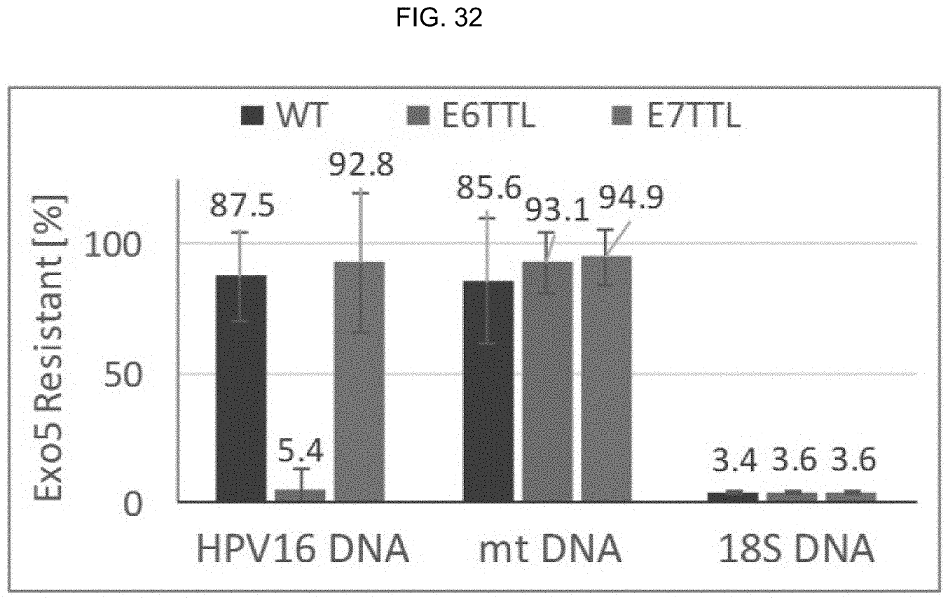

The inventors also subjected HFK infected with mutant and wt HPV16 to long-term culturing to monitor cell survival, viral transcript, and genome levels. Almost complete loss of viral transcripts were observed within 27-33 dpi with E6-TTL mutant virus (FIG. 30). This was accompanied by a loss of viral genome (FIG. 31). In contrast, HFKs infected with the E7-TTL mutant retained high levels of viral transcripts (FIG. 30). To test whether viral genomes were maintained as episomes, the inventors developed an assay to determine the resistance of HPV16 genome to exonuclease 5. Intact double-stranded circular DNA is not a substrate for this enzyme. DNA was isolated from HFK infected with wt, E6-, and E7-TTL mutant virus at 29-33 dpi, treated with exonuclease 5 and subjected to qPCR. 18S ribosomal DNA was completely digested in all samples indeed confirming that the nuclease treatment was sufficient for removal of linear DNA (FIG. 32). In contrast, mitochondrial DNA was mostly resistant as expected for a circular DNA molecule. The inventors found that HPV DNA isolated from cells infected with wt and E7-TTL mutant virus was mostly resistant confirming that they are not substrates for exonucleases and thus likely present as circular DNA. In contrast, the low levels of viral genome still present at late times post infection with E6-TTL mutant virus was sensitive to exonuclease indicating that the remaining viral genome was either integrated or compromised otherwise. Upon long term culturing, HFK infected with E6- and E7-TTL mutant virus as well as mock infected HFK started to senesce approximately 25 to 35 dpi, when they reached the end of their life span. The exact timing varied between different HFK lots used. Wt HPV16-infected HFK, however, continued to grow and express high levels of viral transcripts (FIG. 30). The inventors have cultured these cells for additional 50 days without any sign of senescence, suggesting that they are immortalized. Taken together, these results suggest that neither E6 nor E7 are essential for establishing infection. However, E6 protein is essential for episomal genome maintenance, whereas loss of E7 protein does neither impair genome maintenance nor the viral transcription program in the maintenance stage of infection. However, E7 is distinctly preferable for immortalization of primary HFK under the inventors' conditions.

Discussion

Herein, the inventors describe a novel cell culture system that allows the study of the complete HPV16 life cycle following infectious delivery. Rather than binding virus directly to the cell surface, which restricts uptake by primary keratinocytes for unknown reasons, the inventors used an ECM-to-cell transfer for infection of primary cells. This approach resulted in efficient uptake of viral genome by the majority of cells. Throughout the development of this infection model the inventors used primary cells grown in the presence or absence of the Rho kinase inhibitor Y-27632 and found no significant difference in infection efficiency. Y-27632 promotes immortalization of primary keratinocytes. Taken together, this suggests that immortalization and/or the use of Y-27632 is not essential for increased infection rates. The disclosed model artificially mimics natural infection in that (i) it utilizes pre-binding of virions to the basement membrane equivalent; (ii) only the early but not the late promoter is active in undifferentiated HFK; (iii) early and late promoter are responsive to differentiation triggered by growth in methylcellulose or organotypic raft cultures; (iv) viral genome remains episomal and is amplified upon differentiation; and (v) capsid proteins are expressed in the upper layers of organotypic rafts. At this time, the inventors can only speculate why ECM-to-cell transfer is superior for infecting primary keratinocytes over direct binding to the cell surface. As the inventors' data suggests that the presence of the ECM component LN332 is important for efficient infection, it is assumed that the interaction of the HPV16 capsid with LN332 induces unique conformational changes possibly allowing for direct transfer to the cellular uptake receptor. Furthermore, HFK are polarized and uptake via the basolateral surface may be more efficient than uptake by the apical surface, as appears to be the case for other epitheliotropic viruses.

Most models of the HPV life cycle assume that incoming viral genome is amplified, which is followed by subsequent genome maintenance and low transcriptional activity of viral promoters after infections have been established in the basal cell compartment. They also depict early promoter activation when infected keratinocytes enter terminal differentiation, in addition to the well-studied late promoter activation. It is unclear whether genome amplification requires an initial boost of transcription and whether the shift to genome maintenance is accompanied by early promoter repression. Also, no robust experimental data exist in support of viral genome amplification following infectious delivery. The current cell culture models using immortalized cells do not allow studying the temporal regulation of viral promoters during the immediate early stages of the viral life cycle. In addition, the early promoter is only weakly upregulated upon differentiation. The inventors now find that the p97 early promoter strongly responds to differentiation, which, in turn, suggests that the early promoter is repressed in the basal cells. The inventors also found that the splice variant encoding for E8\hat{ }E2 is the only early transcript whose relative levels increase over time post infection of monolayer cells. E8\hat{ }E2 is a potent inhibitor of viral replication and transcription and restricts viral genome copy numbers in HPV-harboring immortalized cells. E8\hat{ }E2 is transcribed from a recently identified promoter located in the E1 ORF. The E8 promoter has not been studied in great detail, notably, knowledge about its temporal regulation post infectious delivery of viral genome is completely lacking. The infection model will provide a potent platform to study the temporal regulation of the E8 promoter following infectious delivery of viral genome. E8's regulation may allow the E8\hat{ }E2 repressor to orchestrate the shift from establishment of infection, which has been suggested to involve a boost of viral transcription and genome amplification, to maintenance transcription and replication.

Despite extensive studies regarding the functions of early viral proteins in immortalization, transformation and transcriptional regulation, we still know very little about their roles during the viral life cycle; owing mainly to our inability to establish cell lines carrying mutations in many viral genes. The inventors generated HPV16 virions in the HEK 293TT cell line, which does not require HPV factors other than the capsid proteins expressed from a heterologous expression vector. Therefore, the system is amenable to extensive mutational manipulation. As a proof of principle that the infection model will allow investigation of the contributions of individual viral proteins to the complete viral life cycle, the inventors tested E1-, E6-, and E7-TTL mutant viruses for their ability to establish infection and retain episomal genome. As expected, the E1-TTL mutant was unable to efficiently establish infection. Viral transcripts are present, however, at levels 1% below that of wt HPV16 at 6 dpi. In turn, this indirectly suggests that viral genome is amplified following infectious entry. However, it is also conceivable that replication is essential for efficient transcription and further experimentation is required to clarify this point. In contrast, E6- and E7-TTL mutant virus established infection, suggesting they are not essential for immediate early events of the viral life cycle. However, viral transcript levels were consistently lower after infection with E6-compared to E7-TTL mutant and wt virus. Analysis of infected cells at subsequent passages suggests that E6-TTL failed to retain episomal viral genome and viral transcripts were not detectable. Published data using mutants of HPV16 and HPV31 are somewhat conflicting. For HPV31, it was shown that both E6 and E7 were required to establish stably transfected cell lines containing episomal viral genome. In contrast, HPV16 genome harboring E7 mutations were episomally maintained in immortalized NIKS keratinocytes. It is interesting to note that previously described E7-mediated changes to the host cell transcriptome, many of which involve S phase genes, do not seem to be essential for genome maintenance, as the cells infected with E7-TTL mutant virus retain episomal genomes until they senesce. However, the inventors have not yet compared the host transcripts from cells infected with wild type and E7-TTL mutant virus to formally show which alterations to the transcriptome are seen in wild type-infected cells and which of these are due to E7 expression.

The infection model will provide a unique platform to identify host cell factors transcriptionally regulated by the viral oncoproteins after infectious delivery of viral genome without the requirement for immortalization. Analyses of transcripts isolated from individual layers of the stratified epithelia obtained after growth of infected and immortalized HFK as organotypic raft cultures may provide important clues regarding the involvement of altered pathways in the viral life cycle. In future studies, it should be possible to link alterations of the transcriptome to specific functions of the oncoproteins by using mutant viruses. While many of the biological functions and interacting partners of E6 and E7 are identical between low- and high-risk HPV types, it is still not clear, which activities of the high-risk HPV types are ultimately responsible for immortalization. The infection model should be extendable to the study of low-risk HPV types such as HPV6 and 11, which cannot be studied with the current cell culture systems due to their inability to immortalize keratinocytes. A comparative analysis combined with a genetic approach should identify activities absolutely essential for completion of the viral life cycle of both virus groups and may in turn identify functions mediating immortalization. The low-risk HPV types are known not only to cause genital warts but also recurrent respiratory papillomatosis, a debilitating disease requiring repeated surgical procedures, for which no treatment other than surgery is currently available. The extension of the herein described infection model to low-risk HPV types will provide the first platform to investigate and test potential drug candidates for treatment. The infection model may also allow the investigation of skin cancer-linked HPV types from the β-genus and their cooperation with UV irradiation, including the proposed hit and run mechanism of carcinogenesis. The establishment of this infection model will provide a new experimental tool for the study of the HPV life cycle and will help further our understanding of the biological processes leading to immortalization. Furthermore, it will be helpful for the emerging field of studying the synergy of different pathogens in the development of tumors such as oropharyngeal squamous cell carcinoma.

Materials and Methods

Cell Lines.

Human embryonic kidney 293TT and HeLa cells were obtained from John Schiller and Daniel DiMaio, respectively. They were cultured in DMEM supplemented with 10% FBS, non-essential amino acids, antibiotics, and L-Glutamax. Spontaneously immortalized human keratinocytes HaCaT cells were purchased from the American Type Culture Collection (ATCC) and grown in low glucose DMEM containing 5% FBS and antibiotics. Human foreskin keratinocytes (HFKs) were derived from neonatal human foreskin epithelia and maintained in E medium containing mouse epidermal growth factor (EGF) and mitomycin-treated mouse 3T3 J2 fibroblasts. Pooled primary epithelial keratinocytes were also purchased from the ATCC (PCS-200-010) and used in some experiments. In early experiments where indicated, the inventors maintained and infected primary keratinocytes in the presence of the Rho kinase inhibitor (ROCK) Y-27632, which was reported to increase their lifespan. However, the ROCK inhibitor was excluded prior and during experiments involving long term culturing of infected primary cells. Stable cell lines containing HPV16 episomes were created by co-transfection of pEGFP-N1-HPV16 containing the HPV16 genome (W12 strain) with an expression vector for Cre recombinase and a Neomycin resistance plasmid. Cells were transfected using polyethyleneimine (PEI; Polysciences), selected with G418, and expanded as previously described. Episomal maintenance of the viral genome was confirmed using Southern blotting. Differentiation was induced by suspending cells in 1.5% methylcellulose (MC) for 24 hours followed by washes in phosphate buffered saline. Human primary tonsil cells were isolated from tonsils and maintained in E medium with mitomycin-treated mouse 3T3 J2 fibroblasts. Before harvesting RNA or DNA, fibroblast feeders were removed by short trypsin treatment, followed by two washes in PBS.

Ethics Statement.

Foreskin and tonsillar keratinocytes were collected from discarded tissue following routine circumcisions and tonsillectomy from anonymous donors attending University Health, Shreveport. Because the samples were de-identified, would otherwise have been discarded, and were not collected specifically for our studies, the LSUHSC-S IRB ruled that they fell under the NIH's definition of “exempt” from human subjects research, including informed consent (Institutional IRB approval number: STUDY00000187).

Generation of HPV16 Pseudo- and Quasivirions.

The pSheLL16 L1/L2 packaging plasmid and pfwB plasmid, expressing enhanced green fluorescent protein (GFP) were a kind gift from John Schiller, Bethesda, Mass. The plasmid pEGFP-N1 containing the entire floxed HPV16 genome (pEGFP-N1-HPV16) and pBCre plasmid have been described previously (52). Quasivirions were generated using 293TT cells following the improved protocol of Buck and Thompson with minor modifications. Briefly, 293TT cells were first cotransfected with the pSheLL16 L1/L2 and pEGFP-N1-HPV16 plasmids and 24 hours later transfected with the pBCre plasmid. An additional two days later, cells were harvested and viral particles were purified as described previously. Because activity of the Cre recombinase generates two circular plasmids of packable size (pEGFPN1 and HPV16 genome), isolated viral particles comprise a mixture of pseudovirions (pEGFPN1 plasmid) and quasivirions (HPV16 genome). Pseudovirions harboring GFP were also generated in 293TT cells as described by Buck et al. For pseudogenome detection by fluorescence microscopy, pseudogenomes were labeled with EdU (5-ethynyl-2′-deoxyuridine) by supplementing the growth medium with 100 μM EdU at 6 hours post transfection as described during generation of pseudovirions. The viral genome equivalence (vge) was determined by real-time quantitative PCR (RT-qPCR) of encapsidated DNA isolated using the NucleoSpin® Blood QuickPure (Macherey-Nagel; 740569.250).

To introduce a TTL into the E1 ORF, the pEGFP-N1-HPV16 plasmid was digested with ApaI for 1 h at 25 C. The subsequent ˜4500 bp fragment was excised from the gel, purified using DNA gel clean-up kit (Macherey-Nagel, 740609.50), and subcloned into pBlueScript KS II. Next, we used site-directed mutagenesis to substitute a single nucleotide at position 892 within the E1 ORF, which introduced an in-frame TAA ‘stop’ codon just downstream of the E1 start codon. Once confirming the substitution by DNA sequencing, we re-digested the plasmids with ApaI, excised and gel-purified the mutated fragment and vector, and re-ligated it back into the original pEGFP-N1-HPV16 plasmid. The correct insert was again confirmed by sequencing. Primers used: Forward 5′-CCA TGG CTG ATC CTG CAG GTA CCA ATG GGT AAG AGG GTA CGG GAT GTA ATG G-3′, Reverse 5′-CCA TTA CAT CCC GTA CCC TCT TAC CCA TTG GTA CCT GCA GGA TCA GCC ATG G-3′. The E7-TTL mutant has been described previously. Site directed mutagenesis to create TTL mutations in the E6 open reading frame of pEGFP-N1-HPV16 was performed using the QuickChange II Site Directed Mutagenesis kit (Agilent) using primers 5′-GCAATGTTTCAGGACCCATAGTAGTGACCCAGAAAGTTAC-3′ and 5′-GTAACTTTCTGGGTCACTACTATGGGTCCTGAAACATTGC-3′ and confirmed by sequencing.

Infection Using Extracellular Matrix (ECM)-to-Cell Transfer.

HaCaT cells were seeded in 60 mm cell culture dishes and grown for 24-48 h until they reached confluency to allow secretion of ECM. Cells were incubated in Dulbecco's PBS supplemented with 0.5 mM EDTA for up to 2 h in order to remove the cells. To prevent outgrowth of residual HaCaT cells, the dish surface was treated with 8 μg/ml mitomycin for 4 h. Optiprep-purified viral particles (>5×107 vge/dish) diluted in 2 ml E medium were added to the ECM for at least 2 h at 37° C. At this time, 5×105 low passage primary keratinocytes were added. Two hours later approximately 1×105 mitomycin-treated fibroblast feeder cells were added in addition. When different sized culture dishes (ranging from 12 well plates to 100 mm dishes) were used, cell and vge numbers were scaled proportionally to the surface area. Infection was continued for up to 7 days or until cells reached confluency.

Methylcellulose-Induced Differentiation of HFK.

In order to induce differentiation of HFK cells, cells were suspended in methylcellulose at 5 to 7 days post infection with HPV16 quasivirions as described. Samples were collected 24 or 48 hours later. Increased levels of differentiation markers were confirmed by Western Blot and RT qPCR.

Organotypic Raft Cultures.

Organotypic raft cultures generated from HFK cells infected for 5 to 7 days with HPV16 quasivirions were grown as described. Briefly one million keratinocytes were seeded onto the surface of the collagen gel containing fibroblasts feeders. Following attachment, the gel with keratinocytes layer was lifted and placed onto a stainless steel grid in a culture dish. Culture medium was added to the dish so that the keratinocyte/collagen plug was exposed to the air from above and to the medium from below. The medium was changed every other day maintaining the air/fluid interface. Rafts were grown for 14 days and samples were collected for RNA/DNA analysis and immunofluorescent staining and FISH. Rafts generated from uninfected HFK seeded on ECM were used as control.

Immunofluorescence and Fluorescent In Situ Hybridization.

HFK cells were infected with EdU-labeled pseudovirions using ECM-to-cell transfer on glass slides. EdU staining was performed according to the manufacturer's directions. In brief, at the indicated times post infection, cells were washed with PBS and fixed with 4% paraformaldehyde for 15 min at room temperature, washed, permeabilized with 0.5% Triton X-100 in PBS for 10 min, washed, and blocked with 5% goat serum in PBS for 30 min followed by a 30 min incubation with Click-iT® reaction cocktail containing AlexaFluor 555 for EdU-labeled pesudogenome detection. After extensive washing, cells were incubated for 30 min with anti-PML (BETHYL; A301-167A), and anti-laminin A/C (Sigma; SAB4200236) primary antibodies at room temperature, washed again extensively, and subsequently incubated with AlexaFluor488- and AlexaFluor647-tagged secondary antibodies (Molecular Probes; A11029, A21245) for 30 min. After extensive washing with PBS, cells were mounted in ‘Gold Antifade’ containing DAPI (Life Technologies; P3693).

Immunofluorescent Staining and FISH Raft Sections.

HPV16 genomic DNA probes for FISH were prepared by gel purification of the entire HPV16 genome from pUC-HPV16 digested with BamHI and generated using BioNick labeling system according to the manufacturer's protocol (Invitrogen, 18247-015). When mentioned, raft sections were stained for the presence of viral proteins prior to in situ hybridization. Paraffin wax embedded sections were dewaxed in series of xylene and alcohol washes, followed by antigen retrieval using microwave heating at 100° C. in citrate buffer with 0.05% Tween for 20 minutes. Slides were permeablized with 0.5% Triton ×100 for 45 minutes and block with 5% goat serum for 1 h. Primary antibodies:anti-L1-7, anti-E1\hat{ }E4 (a kind gift from J. Doorbar), anti-PCNA (Santa Cruz Biotechnology; sc-7907), anti-p53 (Santa Cruz Biotechnology; sc-126) or anti-MCM7 (Abcam; ab52489) were added for overnight incubation at 4° C. After extensive washing with PBS, sections were incubated for 1 h with AlexaFluor-tagged secondary antibodies (Molecular Probes; A21236; A21245; A11030; A11035) for 1 hour. After extensive PBS washing, sections were fixed and slides were treated with 100 ug/ml RNase A in 2×SSC for 1 hour at 37° C. and for 5 min with micrococcal nuclease (NEB; M0247S). Enzymatic activity was blocked by adding 20 mM EGTA for 5 min. Subsequently, the slides were washed three times with 2×SSC, then dehydrated for 2 min each in 70% EtOH, 80% EtOH and 100% EtOH at room temperature. Slides were then denatured in 70% formamide-2×SSC at 76° C. for 3 minutes, followed by dehydration for 2 min each in 70% EtOH (−20° C.), 80% EtOH and 5 min in 100% EtOH at room temperature. The probe was denatured at 74° C. for 10 minutes prior to hybridization overnight at 37° C. After overnight incubation, the slides were washed multiple times, and tyramide-enhanced fluorescence was carried out according to the manufacturer's instructions (Molecular Probes, T20932). After extensive final washing with PBS, cells were mounted in ‘Gold Antifade’ containing DAPI (Life Technologies; P3693). All IF images were captured by using a Leica CTR6000 fluorescence microscope or by confocal microscopy with a 63× objective using a Leica TCS SP5 Spectral Confocal Microscope and processed with Adobe Photoshop software.

RNA Isolation, cDNA Synthesis, Real-Time qPCR.

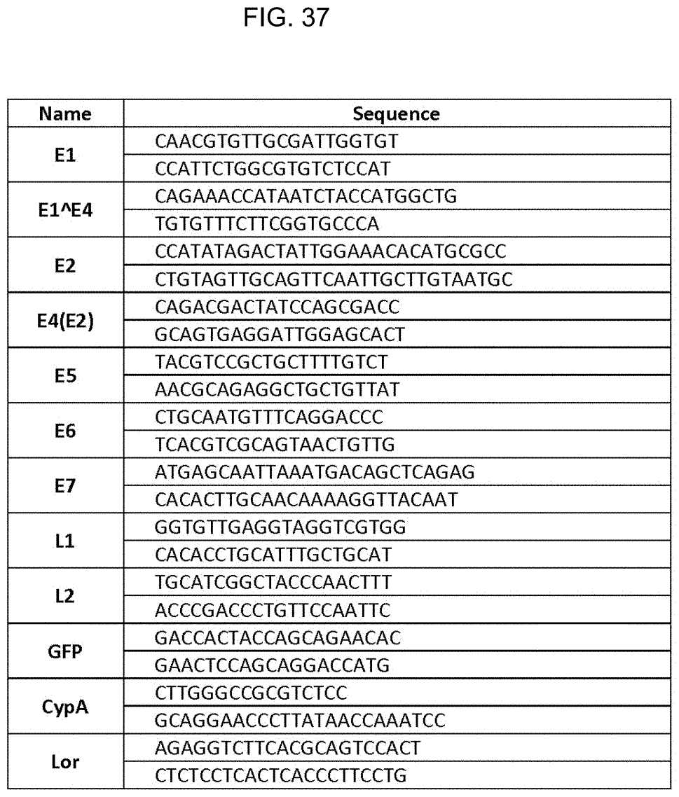

Total RNA from HFK cells was extracted using the RNeasy Plus Mini RNA Isolation Kit (Qiagen; 74236). RNA samples from raft cultures were extracted using RNA Stat-60 (amsbio LLC) according to manufacturer's protocol. Isolated RNA samples were treated with DNase I (NEB; M0303L) prior to reverse transcription. 1 or 0.5 μg total RNA was used to reverse transcribe into cDNA using ImProm-II Reverse Transcriptase kit (Promega). Equal amounts of cDNA were quantified by RT-qPCR using the IQ SYBR Green Supermix (BIO-RAD) and a CFX96 Real-Time System (BIO-RAD). PCR reactions were carried out in triplicate, and transcript levels were normalized to cyclophilin A. Mock reverse inscribed samples were included as negative control. A list of oligonucleotide sequences used is provided in the table shown in FIG. 37. The BIO-RAD CFX Manager 3.1 software was used to analyze the data.

RNA Sequencing.

Total RNA was harvested as described above. RNA quality was assessed on an Agilent Tapestation Bioanalyzer. All samples showed an RNA Integrity Number (RIN) greater than 7. An mRNA sequencing library was prepared with the NEBNextUltra directional library kit and the TruSeq stranded mRNA kit (Illumina). Paired end sequencing (2×75 cycles) was performed on an Illumina NextSeq 500 obtaining over 25 million reads per sample. Reads were aligned to the HPV16 (NC_001526.3) genome using STAR_2.4.2a and counted using RSEM 1.2.31.

Southern Blot.

HFK cells were infected with HPV16 quasivirions using ECM to cell transfer. Uninfected cells served as a control. Genomic DNAs (gDNAs) were isolated from the cells cultured in monolayer for 4 days or cultured in monolayer for 4 days followed by 48 h in methylcellulose. Cell pellets were resuspended in lysis buffer (400 mM NaCl, 10 mM Tris-HCl [pH 7.4], and 10 mM EDTA); then, RNase A (50 μg/ml), proteinase K (50 μg/ml) and 0.2% SDS were added, and the lysates were incubated overnight at 37° C. DNA was extracted with phenol-chloroform and precipitated with ethanol. Approximately 5 □g of gDNA was digested with BgIII (which does not cut the HPV16 genome) and resolved on a 0.8% agarose gel. Genomic DNA fragments were transferred from the gel to DuPont GeneScreenPlus nylon membrane (NEN Research Products, Boston, Mass.) as described by the manufacturer using alkaline transfer. Prehybridization of the membrane was performed for 1 h at 42° C. using a solution containing 50% formamide, 4×SSC, 5×Denhardt's solution, 1% SDS, 10% dextran sulfate, and denatured salmon sperm DNA (0.1 mg/ml). The HPV16 probe was prepared by gel purification of the entire HPV16 genome from pUC HPV16 digested with BamHI and labeling with the Ready-To-Go DNA labeling kit (Amersham Pharmacia). Labeled probe was then purified with ProbeQuant G-50 Micro columns (Amersham Pharmacia), denatured, and added to fresh hybridization solution, which was incubated with membrane at 42° C. overnight. Membrane was washed twice with 2×SSC-0.1% SDS for 15 min at room temperature, twice with 0.5×SSC-0.1% SDS for 15 min at room temperature, twice with 0.1×SSC-0.1% SDS for 15 min at room temperature, and once with 0.1×SSC-1% SDS for 30 min at 50° C. Hybridizing species were visualized by autoradiography.

Western Blot.

Whole-cell extracts were obtained from cell pellets lysed in 1× Laemmli Sample Buffer (BIO-RAD) supplemented with 2-mercaptoethanol. Proteins were resolved on SDS-PAGE and transferred to nitrocellulose membranes (BIO-RAD). Membranes were blocked 1 hour in 5% Blotting-Grade Blocker (BIO-RAD) in 1×TBST) and incubated at 4° C. overnight with anti-cytokeratin 10 (Santa Cruz Biotechnology; sc-52318), anti-E7 (Santa Cruz Biotechnology, sc-6981) or anti-b-actin (Santa Cruz Biotechnology; sc-47778) primary antibodies. After incubation, membranes were washed 3×15 minutes in 1×TBST wash buffer. Membranes were then incubated with horseradish peroxidase-tagged goat anti-mouse and goat anti-rabbit secondary antibodies (1:2500, Jackson ImmunoResearch) at room temperature for 1 hour, washed 3×15 minutes in 1×TBST. Signals were detected by enhanced chemiluminescence (Thermo Scientific). Equal protein loading was confirmed by probing with β-actin monoclonal antibody.

OncoE6™ Cervical Test.

The presence of E6 protein in infected cells was detected using a kit from ArborVita according to the manufacturer's protocol. Briefly, the cell lysate was incubated with alkaline phosphatase conjugated high-affinity E6 HPV16/18 monoclonal antibodies. Next, a nitrocellulose test strip with two capture lines consisting of immobilized mAbs to HPV16/18 E6 was placed into the lysate/mAb-AP mix. The solution was allowed to migrate through the strip by capillary action. E6-mAb-AP present in the sample is forming a ternary complex with the immobilized antibodies on the strip. The complex was visualized as a purple line in the respective location on the strip by the addition of an enzyme substrate solution provided in the kit.

Viral Genome Resistance to Exonuclease 5.

Genomic DNA was isolated using the QIAamp DNA Blood Mini Kit (Qiagen) according to the manufacturer's instructions and stored at 4° C. DNA from UMSCC47 and HPV16-infected 293TT cells served as an HPV16 integration control and episomal HPV16 control, respectively. 100 ng of DNA was either treated with exonuclease V (RecBCD, NEB) or left untreated for 1 hour at 37° C. followed by heat inactivation at 95° C. for 10 minutes. 10 ng of digested/undigested DNA was then quantified by real time PCR using a 7500 FAST Applied Biosystems thermocycler with SYBR Green PCR Master Mix (Applied Biosystems) and 300 nM of each primer in a 15 μl reaction. Nuclease free water was used in place of the template for a negative control. The following cycling conditions were used: 50° C. for 2 minutes, 95° C. for 10 minutes, 40 cycles at 95° C. for 15 seconds, and a dissociation stage of 95° C. for 15 seconds, 60° C. for 1 minute, 95° C. for 15 seconds, and 60° C. for 15 seconds. Separate PCR reactions were performed to amplify HPV16 E6 (1) (F: 5′-GAGAACTGCAATGTTTCAGGACC-3′ R: 5′-TGTATAGTTGTTTGCAGCT CTGTGC-3′), human mitochondrial DNA (2) (F: 5′-CAGGAGTA GGAGAGAGGGAGGTAAG-3′ R: 5′-TACCCATCATAATCGGAGGCTTTGG-3′), and human 18S_ribosomal DNA (3) (F: 5′-GCAATTATTCCCCATG AACG-3′ R: 5′-GGGACTTAATCAACGCAAGC-3′). Human mitochondrial DNA and 18S ribosomal DNA served as episomal and multi-copy linear DNA internal controls, respectively. Primer efficiencies were based on a standard curve generated using a 5-fold dilution series of undigested UMSCC47 DNA and used to calculate the relative amount of DNA per sample. The percent of DNA resistant to exonuclease digestion was calculated relative to undigested DNA.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of comprising:
exposing HPV virions to an extra cellular matrix (ECM) deposition outside of a mammalian body for a first set amount of time.

2. The method of claim 1 further comprising the steps of exposing a mammalian cell to the HPV virion for a second set amount of time.

3. The method of claim 2 wherein the mammalian cell is a human cell.

4. The method of claim 2 wherein the mammalian cell is a keratinocyte.

5. The method of claim 4 wherein the keratinocyte is a primary keratinocyte.

6. The method of claim 1 wherein the HPV virion is one of low-risk HPV and a high-risk HPV.

7. The method of claim 6 wherein the HPV virion a low-risk HPV.

8. The method of claim 7 wherein the low-risk HPV is one of HPV-6, HPV-11, HPV-40, HPV-42, HPV-43, HPV-44, HPV-53, HPV-54, HPV-61, HPV-72, HPV-73, and HPV-81.

9. The method of claim 6 wherein the HPV virion is a high-risk HPV.

10. The method of claim 9 wherein the high-risk HPV is one of HPV-16, HPV-18, HPV-31, HPV-33, HPV-35, HPV-39, HPV-45, HPV-51, HPV-52, HPV-56, HPV-58, HPV-59, and HPV-68.

11. The method of claim 1 wherein the ECM deposition includes depositions secreted by keratinocytes.

12. The method of claim 1 wherein the first set amount of time is at least two hours.

13. The method of claim 2, wherein the second set amount of time is one of greater than 2 days, less than 30 days, and between 2 days and 30 days.

14. The method of claim 2, wherein the second set amount of time is one of greater than 5 days, less than 7 days, and between 5 days and 7 days.

15. The method of claim 2, wherein the second set amount of time is sufficient for the mammalian cells to reach confluency.

16. The method of claim 2 further comprising the step of inducing differentiation of mammalian cells.

17. An HPV virion subjected to the method of claim 1.

18. A mammalian cell subjected to the method of claim 2.

19. The mammalian cell of claim 18, wherein the mammalian cell is a human cell.

20. The mammalian cell of claim 18, wherein the mammalian cell is a keratinocyte.
